(19) United States
(12) Patent Application Publication
(10) Pub. No.: US 2026/0253739 A1
(43) Pub. Date: Aug. 27, 2026

(54) SYSTEM

(71) Applicant: SoftBank Group Corp., Tokyo (JP)

(72) Inventor: Masanori TAKEMURA, Tokyo (JP)

(73) Assignee: SoftBank Group Corp., Tokyo (JP)

(21) Appl. No.: 19/533,376

(22) Filed: Feb. 9, 2026

(30) Foreign Application Priority Data
Feb. 21, 2025 (JP) 2025-027012

(51) Int. Cl. G16H 50/30; G10L 15/16; G10L 25/63; G16H 10/60; G16H 20/70; G16H 40/67; G16H 50/20; H04W 4/029

(52) U.S. Cl. CPC G16H 50/30; G10L 15/16; G10L 25/63; G16H 10/60; G16H 20/70; G16H 40/67; G16H 50/20; H04W 4/029

(57) ABSTRACT

The system according to the embodiment comprises a collection unit, an analysis unit, and an alert unit. The collection unit collects daily activity data of a user. The analysis unit analyzes the data collected by the collection unit and predicts the possibility that the user corresponds to a pre-dementia state. The alert unit issues an alert based on a prediction result obtained by the analysis unit.

### CROSS-REFERENCE TO RELATED APPLICATION

The present application claims priority to and incorporates by reference the entire contents of Japanese Patent Application No. 2025-027012 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, early detection of dementia is difficult, and there is a risk that appropriate treatment to slow its progression may be delayed.

### SUMMARY OF THE INVENTION

The system according to the embodiment comprises a collection unit, an analysis unit, and an alert unit. The collection unit collects daily activity data of a user. The analysis unit analyzes the data collected by the collection unit and predicts the possibility that the user corresponds to a pre-dementia state. The alert unit issues an alert based on a prediction result obtained by the analysis unit.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

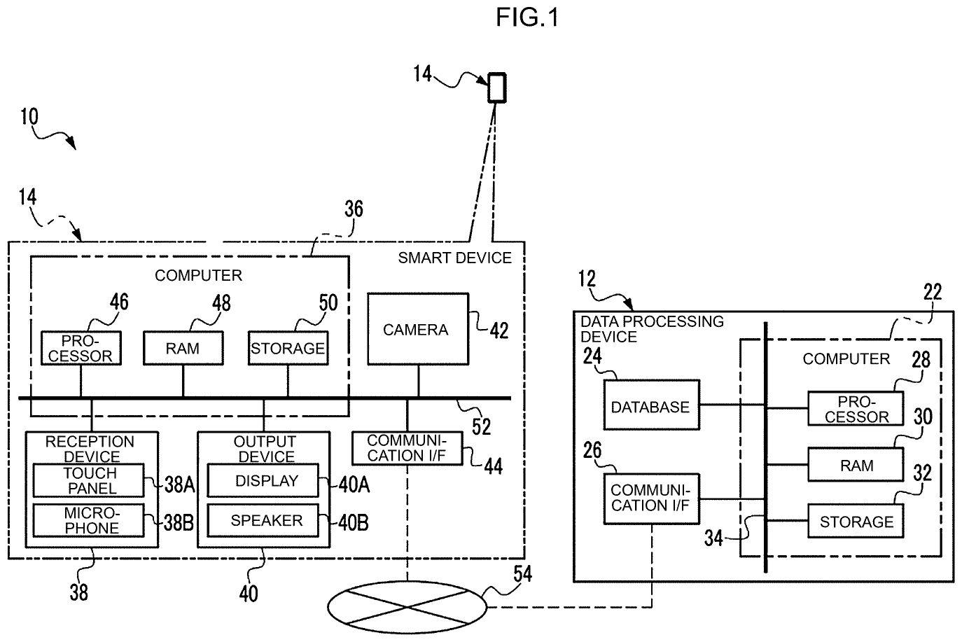

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

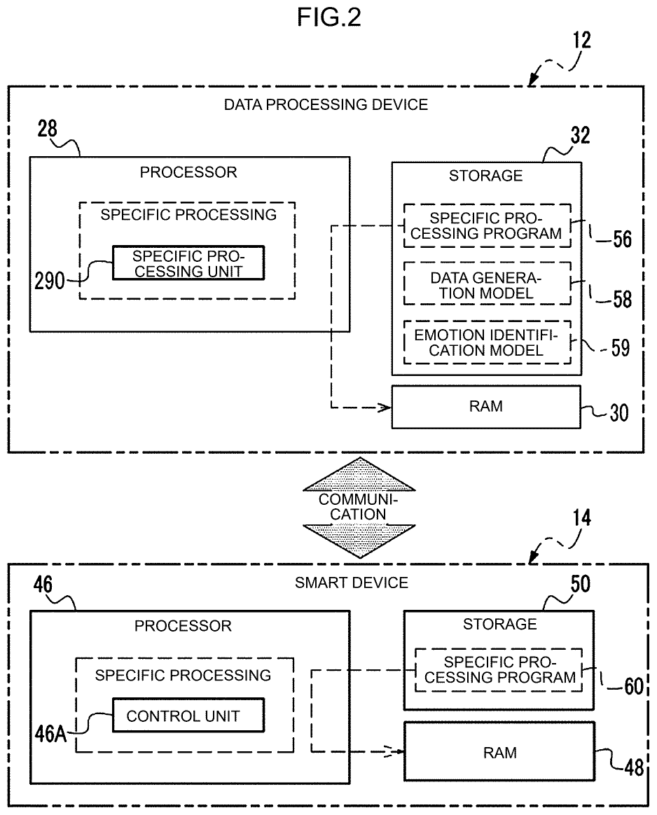

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example of the Embodiment

The system according to the embodiment of the present invention collects behavior data and combines it with AI prediction data to issue alerts regarding the possibility of a pre-dementia state, thereby enabling early treatment. This system collects daily activity data of a user, analyzes it using AI, and predicts the possibility that the user corresponds to a pre-dementia state. Based on the prediction result, if the possibility of a pre-dementia state is high, an alert is issued. Through this alert, the user can visit a medical institution at an early stage and receive appropriate treatment. For example, when collecting daily activity data of a user, various devices are used to collect data such as the user's walking patterns, conversation contents, and meal frequency. This enables detailed understanding of the user's daily activities. Next, the collected behavior data is analyzed by AI. The AI analyzes the user's behavior patterns based on the collected data and predicts the possibility of a pre-dementia state. For example, changes in walking patterns, changes in conversation contents, and changes in meal frequency are analyzed to detect signs of dementia. This allows prediction of the possibility of a pre-dementia state from the user's behavior data. Based on the prediction result, if the possibility of a pre-dementia state is high, an alert is issued. For example, the notification function is used to issue an alert to the user. The alert includes information that there is a possibility of a pre-dementia state and recommends early consultation with a medical institution. As a result, the user can visit a medical institution early and receive appropriate treatment. This enables early detection and early treatment of dementia. The user can grasp signs of dementia based on their own behavior data and take measures at an early stage. For example, by noticing changes in walking patterns or conversation contents and visiting a medical institution, the progression of dementia can be delayed. In addition, family members and caregivers can share the user's behavior data, notice signs of dementia early, and take appropriate actions. Thus, by collecting behavior data and combining it with AI prediction data, it is possible to issue alerts regarding the possibility of a pre-dementia state and lead to early treatment. Specifically, the system collects daily activity data of the user such as walking patterns (e.g., number of steps per day, walking speed, time-series acceleration data of walking rhythm: one-dimensional array, sampling frequency 50 Hz, about 4.32 million samples per day), conversation contents (e.g., speech data recorded at 16 kHz and converted to text by a speech recognition engine, average of 100 utterances per day), and meal frequency (e.g., three meal events per day from a meal recording app, with meal contents, time, and calorie information attached to each event). These data are automatically acquired by the collection unit from various sensor devices and applications and stored in a database in time series. The AI analysis unit uses these multidimensional data as input and, for example, applies convolutional neural networks (CNN), recurrent neural networks (RNN), or Transformer-based time-series analysis models to automatically extract abnormalities in walking patterns (e.g., decrease in walking speed, disruption of rhythm), changes in conversation contents (e.g., decrease in vocabulary, delay in utterance, decrease in semantic consistency), and changes in meal frequency (e.g., decrease in meal count, irregular meal times). Examples of input to the AI include (1) one day's walking acceleration data (50 Hz×86,400 seconds=4.32 million points in a one-dimensional array), (2) one day's utterance text (e.g., “The weather is nice today”, “I ate rice”, about 100 utterances), and (3) one week's meal events (structured data with meal contents, calories, and time attached to each event). The AI output consists of (a) pre-dementia risk score (continuous value from 0.0 to 1.0), (b) risk factor labels (e.g., “abnormal walking pattern”, “decline in conversation contents”), and (c) recommended actions (e.g., “recommend visiting a medical institution”, “follow-up observation”) as structured data. For example, when inputting one week's data for a user, the AI generates output such as “risk score 0.82”, “factors: decreased walking speed, reduced vocabulary in conversation”, “recommendation: visit a medical institution”. The alert unit receives this output and uses smartphone notification functions, email, or voice notification APIs to issue specific alerts to the user or family, such as “There is a high possibility of a pre-dementia state, so please visit a medical institution early”. As a subsequent process, the user's response after the alert is issued (e.g., whether or not a medical institution was visited, sharing with family) is recorded and used as training data for future AI learning. As a technical effect, this system does not depend on manual observation or interviews by humans, but automatically analyzes vast time-series data in high-dimensional space and applies unconventional feature extraction rules (e.g., local pattern detection by CNN, long-term dependency capture by RNN), enabling more accurate and earlier detection of signs of dementia than conventional methods. This reduces the diagnostic burden on medical institutions and enables users and families to take early action voluntarily. Application fields include monitoring of elderly people at home, health management in nursing care facilities, remote medical support, and corporate health management programs. Furthermore, by aggregating anonymized data from multiple users, it is possible to create dementia risk maps at the regional level and apply them to public health policies.

The pre-dementia detection system according to the embodiment comprises a collection unit, an analysis unit, and an alert unit. The collection unit collects daily activity data of a user. The daily activity data of the user may include, for example, walking patterns, conversation contents, and meal frequency, but is not limited to such examples. The collection unit collects the user's walking patterns using, for example, pedometers or acceleration sensors. The collection unit can also collect the user's conversation contents using speech recognition technology. Furthermore, the collection unit can collect the user's meal frequency using a meal recording application. For example, the collection unit records the user's daily number of steps using a pedometer and analyzes the rhythm and speed of walking using an acceleration sensor. Speech recognition technology converts the user's conversation contents into text data and analyzes the contents of the conversation using natural language processing technology. The meal recording application records meal frequency by allowing the user to input meal contents and count. The analysis unit analyzes the data collected by the collection unit and predicts the possibility that the user corresponds to a pre-dementia state. The analysis unit analyzes the collected data using, for example, AI. The AI analyzes the user's behavior patterns based on the collected data and detects signs of dementia. For example, the AI analyzes changes in walking patterns, changes in conversation contents, and changes in meal frequency to predict the possibility of a pre-dementia state. The AI analyzes changes in walking rhythm and speed to detect changes in walking patterns, analyzes changes in conversation contents and tone to detect changes in conversation, and analyzes changes in meal count and contents to detect changes in meal frequency. The alert unit issues alerts based on the prediction result obtained by the analysis unit. The alert unit issues alerts using, for example, smartphone notification functions, email, or voice notifications. The alert includes information that there is a possibility of a pre-dementia state and recommends early consultation with a medical institution. For example, the alert unit issues alerts to the user using smartphone notification functions. Alerts can also be issued using email or voice notifications. Thus, the pre-dementia detection system according to the embodiment enables early detection and early treatment of pre-dementia by collecting, analyzing, and issuing alerts based on the user's daily activity data. Specifically, the pre-dementia detection system automatically acquires multidimensional data such as the number of steps per day from pedometers or acceleration sensors (e.g., 10,000 steps per day), walking speed (e.g., 1.2 m/s), time-series acceleration data of walking rhythm (e.g., 50 Hz sampling, about 4.32 million samples per day), utterance text converted by a speech recognition engine (e.g., 100 utterances per day), and meal events from a meal recording app (e.g., three times per day, with meal contents, time, and calorie information attached to each event), and stores them in a database in time series. The analysis unit uses these data as input and applies AI architectures such as convolutional neural networks (CNN), recurrent neural networks (RNN), and Transformer-based time-series analysis models to automatically extract abnormalities in walking patterns (e.g., decrease in walking speed, disruption of rhythm), changes in conversation contents (e.g., decrease in vocabulary, delay in utterance, decrease in semantic consistency), and changes in meal frequency (e.g., decrease in meal count, irregular meal times). Examples of input to the AI include one day's walking acceleration data (50 Hz×86,400 seconds=4.32 million points in a one-dimensional array), one day's utterance text (e.g., “The weather is nice today”, “I ate rice”, about 100 utterances), and one week's meal events (structured data with meal contents, calories, and time attached to each event). The AI output consists of a pre-dementia risk score (continuous value from 0.0 to 1.0), risk factor labels (e.g., “abnormal walking pattern”, “decline in conversation contents”), and recommended actions (e.g., “recommend visiting a medical institution”, “follow-up observation”) as structured data. For example, when inputting one week's data for a user, the AI generates output such as “risk score 0.82”, “factors: decreased walking speed, reduced vocabulary in conversation”, “recommendation: visit a medical institution”. The alert unit receives this output and uses smartphone notification functions, email, or voice notification APIs to issue specific alerts to the user or family, such as “There is a high possibility of a pre-dementia state, so please visit a medical institution early”. As a subsequent process, the user's response after the alert is issued (e.g., whether or not a medical institution was visited, sharing with family) is recorded and used as training data for future AI learning. As a technical effect, this system does not depend on manual observation or interviews by humans, but automatically analyzes vast time-series data in high-dimensional space and applies unconventional feature extraction rules (e.g., local pattern detection by CNN, long-term dependency capture by RNN), enabling more accurate and earlier detection of signs of dementia than conventional methods. This reduces the diagnostic burden on medical institutions and enables users and families to take early action voluntarily. Application fields include monitoring of elderly people at home, health management in nursing care facilities, remote medical support, and corporate health management programs. Furthermore, by aggregating anonymized data from multiple users, it is possible to create dementia risk maps at the regional level and apply them to public health policies.

The collection unit can collect data such as the user's walking patterns, conversation contents, and meal frequency. The collection unit collects the user's walking patterns using, for example, pedometers or acceleration sensors. For example, the collection unit records the user's daily number of steps using a pedometer and analyzes the rhythm and speed of walking using an acceleration sensor. The collection unit can also collect the user's conversation contents using speech recognition technology. For example, the collection unit converts the user's conversation contents into text data using speech recognition technology and analyzes the contents of the conversation using natural language processing technology. Furthermore, the collection unit can collect the user's meal frequency using a meal recording application. For example, the collection unit records meal frequency by allowing the user to input meal contents and count using a meal recording application. Thus, the collection unit can collect detailed daily activity data of the user and more accurately grasp signs of dementia. Specifically, the collection unit automatically acquires data such as the number of steps per day from a pedometer (e.g., 10,000 steps per day), time-series data of walking rhythm from an acceleration sensor (e.g., 50 Hz sampling, 4.32 million samples per day), utterance text converted by a speech recognition engine (e.g., 100 utterances per day), and meal events from a meal recording app (e.g., three times per day, with meal contents, time, and calorie information attached to each event), and stores them in a database in time series. When collecting these data, the collection unit can automatically detect missing data or outliers and perform preprocessing such as outlier removal or normalization. Furthermore, the collection unit integrates data from multiple sensor devices and applications and manages them with a unique ID for each user to ensure data consistency and reliability. As a technical effect, compared to manual recording or memory-dependent recording by humans, the collection unit can automatically collect vast time-series data at high frequency and high accuracy, enabling earlier and more objective detection of signs of dementia than conventional methods. Application fields include monitoring of elderly people at home, health management in nursing care facilities, remote medical support, and corporate health management programs.

The analysis unit can analyze the user's behavior patterns based on the collected data and detect signs of dementia. The analysis unit analyzes the collected data using, for example, AI. The AI analyzes the user's behavior patterns based on the collected data and detects signs of dementia. For example, the AI analyzes changes in walking patterns, changes in conversation contents, and changes in meal frequency to predict the possibility of a pre-dementia state. The AI analyzes changes in walking rhythm and speed to detect changes in walking patterns, analyzes changes in conversation contents and tone to detect changes in conversation, and analyzes changes in meal count and contents to detect changes in meal frequency. Thus, the analysis unit can detect signs of dementia at an early stage by analyzing the collected data. Specifically, the analysis unit inputs time-series acceleration data of walking patterns (e.g., 50 Hz sampling, 4.32 million samples per day), utterance text (e.g., 100 utterances per day), and meal event data (e.g., structured data including meal contents, calories, and time for one week) into an AI model. The analysis unit uses convolutional neural networks (CNN) to extract local abnormal patterns (e.g., decrease in walking speed, disruption of rhythm) from walking data and uses recurrent neural networks (RNN) or Transformer-based models to detect long-term behavioral changes. The analysis unit uses natural language processing algorithms (e.g., BERT or LSTM) to quantitatively evaluate vocabulary, semantic consistency, and utterance delay in conversation contents. For meal data, the analysis unit automatically extracts decreases in meal count, irregular meal times, and nutritional imbalance. Examples of input to the AI include one day's walking acceleration data (one-dimensional array), one day's utterance text (100 utterances), and one week's meal events (structured data). The AI output consists of a pre-dementia risk score (0.0 to 1.0), risk factor labels (e.g., “abnormal walking pattern”, “decline in conversation contents”), and recommended actions (e.g., “recommend visiting a medical institution”) as structured data. The analysis unit uses these outputs to perform threshold judgment and branching processing, and utilizes them for notification to the alert unit and accumulation of subsequent learning data. As a technical effect, the analysis unit does not depend on subjective observation or interviews by humans, but automatically analyzes vast multidimensional data in high-dimensional space and applies unconventional feature extraction rules, enabling more accurate and earlier detection of signs of dementia than conventional methods. Application fields include monitoring of elderly people at home, health management in nursing care facilities, and remote medical support.

The alert unit can issue alerts using smartphone notification functions, email, or voice notifications. The alert unit issues alerts to the user using, for example, smartphone notification functions. For example, the alert unit issues alerts to the user using smartphone notification functions. Alerts can also be issued using email. For example, the alert unit issues alerts to the user using email. Alerts can also be issued using voice notifications. For example, the alert unit issues alerts to the user using voice notifications. Thus, by issuing alerts, the alert unit can prompt the user to visit a medical institution at an early stage. Specifically, the alert unit automatically generates alert messages based on structured data received from the analysis unit, such as pre-dementia risk score (e.g., 0.82), risk factor labels (e.g., “decreased walking speed”, “reduced vocabulary in conversation”), and recommended actions (e.g., “recommend visiting a medical institution”). The alert unit considers the user's attribute information (e.g., age, smartphone usage status) and current geographic location information (e.g., home, outside) to select the optimal notification means (e.g., smartphone notification, email, voice notification API). After issuing an alert, the alert unit records the user's response (e.g., notification confirmation, whether or not a medical institution was visited) and uses it as training data for future AI learning. The alert unit can also simplify or detail the alert content according to the user's emotional state (e.g., stress, relaxation) or situation (e.g., when in a hurry). As a technical effect, the alert unit can promptly and appropriately convey important health information to users and families, thereby promoting early visits to medical institutions and voluntary health actions, contributing to suppression of dementia progression and reduction of medical burden. Application fields include monitoring of elderly people at home, health management in nursing care facilities, remote medical support, and corporate health management programs.

The collection unit anonymizes the collected data and does not provide it to third parties. The collection unit anonymizes the collected data, for example. For example, the collection unit deletes personal information from the collected data and masks the data. As a result, the collected data is anonymized and not provided to third parties. The collection unit ensures that data is not provided to third parties by, for example, establishing data management methods and access restrictions. Thus, anonymization of the collected data enables protection of user privacy. Specifically, the collection unit removes personal identification information such as user ID, name, address, and contact information from the database and assigns a randomly generated unique identifier (e.g., UUID) to each user. The collection unit manages behavior data such as walking patterns, conversation contents, and meal events linked to anonymized IDs so that individuals cannot be identified. The collection unit strictly controls access rights to the database to ensure that third parties other than administrators or the analysis unit cannot access the data. Furthermore, the collection unit uses encrypted communication (e.g., TLS) during data transfer to reduce the risk of unauthorized access or leakage from outside. As a technical effect, the collection unit can highly protect user privacy while safely performing AI analysis and group statistical processing using anonymized data, thereby contributing to improved user reliability and legal compliance (e.g., GDPR). Application fields include utilization of data in medical and nursing care fields, creation of health risk maps at the regional level, and application to public health policies.

The collection unit can estimate the user's emotions and adjust the timing of collecting behavior data based on the estimated emotions. The collection unit estimates the user's emotions, for example. For example, the collection unit estimates the user's emotions using facial expression recognition technology. The collection unit adjusts the timing of collecting behavior data based on the user's emotions, for example. For example, if the user is feeling stressed, the collection unit reduces the collection timing to lessen the user's burden. If the user is relaxed, the collection unit increases the collection timing to collect more detailed data. If the user is in a hurry, the collection unit adjusts the collection timing to collect only important data. Thus, by adjusting the collection timing according to the user's emotions, the collection unit can reduce the user's burden. Specifically, the collection unit uses facial image data (e.g., 128×128 pixel RGB images, acquired at one frame per second), audio data (e.g., one-second audio waveform sampled at 16 kHz), and biometric sensor data (e.g., heart rate, skin conductance as one-dimensional time-series data) as input for emotion estimation. The collection unit inputs these data into convolutional neural networks (CNN), recurrent neural networks (RNN), or multimodal Transformer models to output emotion classification labels (e.g., “stress”, “relaxation”, “in a hurry”) and emotion intensity scores (continuous values from 0.0 to 1.0). For example, when facial images and audio waveforms are input simultaneously, the collection unit outputs a probability distribution such as “stress: 0.78”, “relaxation: 0.12”, “in a hurry: 0.10”. Based on these output values, the collection timing control module automatically applies rule-based scheduling such as “10-minute intervals when stress is high, 1-minute intervals when relaxed, immediate collection of important data when in a hurry”. The collection unit monitors emotion estimation results in real time and automatically reduces collection frequency when a threshold (e.g., stress score of 0.7 or higher) is exceeded. The collection unit implements an optimal collection timing algorithm (e.g., parameter auto-adjustment by reinforcement learning) according to emotional state to minimize user burden while ensuring necessary data. As a technical effect, the collection unit can maximize data collection efficiency without impairing user experience by relying on high-precision emotion estimation and automatic scheduling by AI, rather than subjective human judgment or manual settings. This enables stable acquisition of high-quality behavior data for pre-dementia detection while greatly reducing user stress and burden. Application fields include monitoring of elderly people at home, health management in nursing care facilities, remote medical support, and health management programs involving stress management. Furthermore, by statistically analyzing the relationship between emotional states and collection frequency for multiple users, it is possible to apply to group optimization and personalized health support.

The collection unit can analyze the user's past behavior data and select an optimal collection method. The collection unit analyzes the user's past behavior data, for example. For example, the collection unit analyzes past records or historical data. The collection unit customizes the collection method based on actions frequently performed by the user in the past, for example. The collection unit selects the most efficient collection method based on the user's past behavior data, for example. The collection unit optimizes the collection method by analyzing the user's past behavior patterns, for example. Thus, by analyzing past behavior data, the collection unit can select an optimal collection method. Specifically, the collection unit uses behavior data accumulated in time series for each user (e.g., numerical vectors such as daily steps, number of conversations, number of meal events, sleep time, for one week to one year of history) as input. The collection unit inputs these historical data into time-series analysis models (e.g., LSTM, GRU, autoregressive models) or clustering algorithms (e.g., k-means, DBSCAN) to automatically extract the user's behavior patterns (e.g., differences in activity between weekdays and holidays, seasonal variations, changes during specific events). Based on the extracted patterns, the collection unit optimizes collection frequency and types of data to be collected in a personalized manner. For example, if user A has taken a walk at the same time every day for the past month, the collection unit increases the sampling frequency of walking data during that time. Conversely, if user B tends to have more conversations only on holidays, the collection unit strengthens conversation data collection on holidays. Based on AI pattern recognition results, the collection method selection module automatically sets “collection frequency, target sensors, and data granularity for each day of the week, time slot, and event type”. The collection unit continuously updates parameters to maximize collection efficiency (e.g., data amount/user burden ratio) using optimization algorithms (e.g., Bayesian optimization, reinforcement learning). As a technical effect, the collection unit can realize an optimal data collection strategy for each user by relying on AI-based historical analysis and automatic optimization, rather than human heuristics or uniform settings. This enables efficient acquisition of information necessary for pre-dementia detection while reducing unnecessary data collection and user burden. Application fields include individual health management, personalized medicine, behavior change support, and operational efficiency improvement in nursing care settings. Furthermore, by aggregating historical data from multiple users and deriving optimal collection strategies for each group, it is possible to apply to large-scale data collection optimization at the regional or facility level.

The collection unit can perform filtering based on the user's current health condition and living environment when collecting behavior data. The collection unit evaluates the user's health condition, for example. For example, the collection unit evaluates the user's health condition based on medical data or self-reports. The collection unit evaluates the user's living environment, for example. For example, the collection unit evaluates the user's living environment based on housing environment or lifestyle habits. The collection unit filters collected data based on the user's health condition and living environment, for example. For example, if the user's health condition is poor, the collection unit reduces the amount of collected data to lessen the user's burden. If the user's living environment changes, the collection unit collects data adapted to the new environment. The collection unit filters collected data based on the user's health condition and living environment, for example. Thus, by filtering data based on the user's health condition and living environment, the collection unit can reduce the user's burden. Specifically, the collection unit uses medical data indicating the user's health condition (e.g., diagnostic history from electronic medical records, medication information, self-reported health scores) and living environment data (e.g., housing type, number of cohabitants, results of lifestyle habit questionnaires as structured data) as input. The collection unit inputs these data into rule-based filtering engines or decision tree models and applies dynamic collection control rules such as “halve walking data collection frequency when health condition is poor” or “prioritize collection of new behavior patterns when living environment changes”. For example, if the user is hospitalized, the collection unit stops collecting walking data or outdoor activity data and instead strengthens collection of indoor activity or sleep data. If the user's living environment changes due to moving or changes in cohabitants, the collection unit focuses on collecting data adapted to the new environment (e.g., new daily rhythm, meal patterns). The collection unit monitors changes in health condition and living environment in real time and automatically adjusts collection targets, frequency, and granularity according to threshold judgments (e.g., health score below a certain value) or event triggers (e.g., living environment change flag). As a technical effect, the collection unit realizes flexible data collection control according to individual user situations, minimizes unnecessary data collection and user burden, and reliably acquires information necessary for pre-dementia detection. This enables both data quality and user experience compared to conventional uniform collection methods. Application fields include chronic disease management, home medical care, operational efficiency improvement in nursing care settings, and personalized health support. Furthermore, by group analysis of patterns of changes in health condition and living environment, it is possible to apply to optimization of public health policies and regional medical resource allocation.

The collection unit can estimate the user's emotions and determine the priority of behavior data to be collected based on the estimated emotions. The collection unit estimates the user's emotions, for example. For example, the collection unit estimates the user's emotions using facial expression recognition technology. The collection unit determines the priority of behavior data to be collected based on the user's emotions, for example. For example, if the user is feeling stressed, the collection unit prioritizes collection of only important data. If the user is relaxed, the collection unit prioritizes collection of detailed data. If the user is in a hurry, the collection unit adjusts the priority of data to be collected. Thus, by determining the priority of collected data according to the user's emotions, the collection unit can prioritize collection of important data. Specifically, the collection unit uses facial image data (e.g., 128×128 pixel RGB images), audio data (e.g., audio waveform sampled at 16 kHz), and biometric sensor data (e.g., heart rate, skin conductance) as input for emotion estimation. The collection unit inputs these data into multimodal AI models (e.g., combination of CNN and RNN, or Transformer-based emotion estimation models) to output emotion labels (e.g., “stress”, “relaxation”, “in a hurry”) and emotion intensity scores. Based on emotion estimation results, the collection unit implements a priority determination module to determine the priority of target data to be collected (e.g., walking patterns, conversation contents, meal events, sleep data, etc.). For example, when stress is high, only walking patterns and sleep data are collected; when relaxed, all data are collected in detail; when in a hurry, only conversation contents and meal events are collected, according to rule-based settings. The collection unit uses weighted scoring or decision trees as priority determination algorithms and automatically adjusts collection targets, frequency, and granularity according to emotional state. The collection unit records the results of prioritization in real time and utilizes them for subsequent AI analysis and optimization of user experience. As a technical effect, the collection unit can reliably acquire information necessary for pre-dementia detection while minimizing user burden by prioritizing collection of important data according to the user's emotional state. This enables both data quality and user experience compared to conventional uniform collection methods. Application fields include monitoring of elderly people at home, health management in nursing care facilities, health management programs involving stress management, and personalized health support. Furthermore, by group analysis of the relationship between emotional state and data priority, it is possible to apply to group optimization and behavior change support.

The collection unit can preferentially collect highly relevant data by considering the user's geographic location information when collecting behavior data. The collection unit collects the user's geographic location information, for example. For example, the collection unit collects the user's geographic location information using GPS data or location information services. The collection unit preferentially collects highly relevant data based on the user's geographic location information, for example. For example, if the user is in a specific location, the collection unit preferentially collects data related to that location. The collection unit collects highly relevant data based on the user's geographic location information, for example. If the user is moving, the collection unit preferentially collects data related to the destination. Thus, by considering the user's geographic location information, the collection unit can preferentially collect highly relevant data. Specifically, the collection unit uses GPS data acquired from the user's smartphone or wearable device (e.g., time-series data with latitude, longitude, altitude, and timestamp, 24 hours at one-minute intervals) and Wi-Fi/Bluetooth beacon information as input. The collection unit matches these location data with a map database and automatically assigns location labels such as “home”, “workplace”, “park”, “medical institution”. The collection unit implements a rule-based engine or decision tree to set the priority of data to be collected for each location label and dynamically controls collection, such as “prioritize sleep and meal data at home, walking patterns at the park, health condition data at medical institutions”. If the user is moving, the collection unit analyzes the movement route and speed and focuses on collecting data related to the destination (e.g., conversation contents at the destination, walking patterns while moving). The collection unit scores the relevance between location information and behavior data using AI models (e.g., random forest, gradient boosting) and preferentially collects data with high relevance scores. As a technical effect, the collection unit can automatically apply the optimal data collection strategy according to the user's geographic location, suppress unnecessary data collection, and efficiently acquire useful information for pre-dementia detection. This enables both data quality improvement and reduction of user burden. Application fields include monitoring of elderly people at home, health management during outings, behavior monitoring in nursing care facilities, and regional medical collaboration. Furthermore, by group analysis of patterns of relevance between location information and behavior data, it is possible to create health risk maps at the regional level and apply them to public health policies.

The collection unit can analyze the user's social media activities when collecting behavior data and collect relevant data. The collection unit analyzes the user's social media activities, for example. For example, the collection unit analyzes post contents and activity frequency. The collection unit analyzes the user's social media activities and collects relevant behavior data, for example. The collection unit selects data to be collected based on information shared by the user on social media, for example. The collection unit collects highly relevant data from the user's social media activities, for example. Thus, by analyzing the user's social media activities, the collection unit can collect highly relevant data. Specifically, the collection unit automatically acquires public post data via API from multiple social media platforms used by the user (e.g., text posting, image sharing, video streaming platforms). The collection unit inputs acquired post data (e.g., text content, posting time, posting frequency, image metadata, video view count as structured data) into natural language processing engines or image analysis AI models. For text posts, the collection unit uses language models such as BERT or Transformer-based models to extract features such as emotional tendency (e.g., “positive”, “negative”, “neutral”), topic classification (e.g., “health”, “hobbies”, “family”), vocabulary, and grammatical complexity. For image posts, the collection unit uses CNN-based image classification models to automatically determine image content (e.g., “outdoor activity”, “meal”, “group activity”), color tone, and number of subjects. For activity frequency, the collection unit uses time-series analysis models (e.g., LSTM) to extract patterns such as number of posts per day and variations by day of the week or time of day. Examples of input to the AI include (1) one week's text posts (content, posting time, emotion score for each post), (2) image post metadata (image feature vector, posting time), and (3) time-series data of posting frequency (vector of number of posts per day). The AI output consists of (a) social activity score (continuous value from 0.0 to 1.0, e.g., 0.85 is active, 0.20 is low activity), (b) activity tendency labels (e.g., “decline in activity”, “increase in health topics”, “tendency toward isolation”), and (c) collection priority (e.g., “prioritize outdoor activity data”, “prioritize conversation data”) as structured data. For example, if the user's posting frequency halves in one month and post contents become biased toward negative tendencies such as “loneliness” or “fatigue”, the collection unit assigns an “activity decline” label and strengthens collection of outdoor activity and conversation data. The collection unit automatically adjusts the targets, frequency, and granularity of behavior data collection based on these AI outputs. As a subsequent process, if changes in social media activity are detected, the collection unit notifies the analysis unit or alert unit and uses it as a trigger for pre-dementia risk assessment or alert issuance. As a technical effect, the collection unit can detect changes in social activity or tendencies toward isolation with high accuracy and at an early stage by combining high-dimensional feature extraction and multifaceted analysis by AI, rather than relying on subjective human observation or simple post count. This enables objective understanding of social isolation and activity decline, which are risk factors for pre-dementia, based on data and realization of appropriate data collection and intervention. Application fields include monitoring of elderly people at home, social activity monitoring in nursing care facilities, remote medical support, corporate health management programs, and creation of isolation risk maps at the regional level. Furthermore, by aggregating anonymized social activity data from multiple users, it is possible to apply to group-level social activity tendencies and public health policies.

The analysis unit can estimate the user's emotions and adjust the expression method of analysis based on the estimated emotions. The analysis unit estimates the user's emotions, for example. For example, the analysis unit estimates the user's emotions using facial expression recognition technology. The analysis unit adjusts the expression method of analysis based on the user's emotions, for example. For example, if the user is relaxed, the analysis unit provides detailed analysis results. If the user is feeling stressed, the analysis unit provides concise analysis results. If the user is in a hurry, the analysis unit provides analysis results that focus on key points. Thus, by adjusting the expression method of analysis according to the user's emotions, the analysis unit can provide analysis results that are easy for the user to understand. Specifically, the analysis unit uses facial image data (e.g., 128×128 pixel RGB images, acquired at one-second intervals), audio data (e.g., one-second audio waveform sampled at 16 kHz), and biometric sensor data (e.g., heart rate, skin conductance as one-dimensional time-series data) as input for emotion estimation. The analysis unit inputs these data into multimodal AI models (e.g., combination of CNN and RNN, or Transformer-based emotion estimation models) to output emotion labels (e.g., “stress”, “relaxation”, “in a hurry”) and emotion intensity scores (continuous values from 0.0 to 1.0). For example, when facial images and audio waveforms are input simultaneously, the analysis unit outputs a probability distribution such as “stress: 0.72”, “relaxation: 0.18”, “in a hurry: 0.10”. Based on emotion estimation results, the analysis result expression method determination module automatically applies rule-based output control such as “present only key points concisely when stress is high, present with detailed graphs and explanations when relaxed, present only key points in bullet points when in a hurry”. The analysis unit automatically selects the presentation format of analysis results (e.g., text summary, detailed graphs, infographics, audio explanations) according to the user's emotional state. Examples of AI output include (1) “stress state: present only key points (e.g., risk score 0.82, recommend visiting a medical institution)”, (2) “relaxation state: detailed analysis (e.g., graph of walking speed trends, changes in vocabulary in conversation contents, detailed explanation of meal patterns)”, and (3) “in a hurry: bullet point summary (e.g., risk factors, recommended actions only)”. As a subsequent process, after presenting analysis results, the analysis unit records the user's response (e.g., content confirmation, request for detailed display) and uses it for optimization of analysis expression in future sessions. As a technical effect, the analysis unit can maximize understanding and utilization efficiency of analysis results without impairing user experience by relying on high-precision emotion estimation and automatic output control by AI, rather than subjective human judgment or uniform output. This enables reliable transmission of important information for pre-dementia detection while reducing user stress and burden. Application fields include monitoring of elderly people at home, health management in nursing care facilities, remote medical support, and health management programs involving stress management. Furthermore, by statistically analyzing the relationship between emotional state and analysis expression for multiple users, it is possible to apply to group optimization and personalized health support.

The analysis unit can adjust the level of detail of analysis based on the importance of behavior data during analysis. The analysis unit evaluates the importance of behavior data, for example. For example, the analysis unit evaluates the importance of behavior data based on data impact or analysis priority. The analysis unit adjusts the level of detail of analysis based on the importance of behavior data, for example. For example, the analysis unit performs detailed analysis for important behavior data. For behavior data of low importance, the analysis unit performs concise analysis. The analysis unit adjusts the level of detail of analysis according to the importance of behavior data, for example. Thus, by adjusting the level of detail of analysis according to the importance of behavior data, the analysis unit can perform efficient analysis. Specifically, the analysis unit uses multidimensional behavior data received from the collection unit (e.g., time-series acceleration data of walking patterns, text data of conversation contents, structured data of meal events, sleep data, social media activity data) as input. The analysis unit uses AI models (e.g., random forest, gradient boosting, attention mechanism neural networks) to calculate importance scores (continuous values from 0.0 to 1.0) for each data type. For example, if abnormalities in walking patterns strongly contribute to dementia risk, “walking data importance 0.92” is evaluated; if changes in conversation contents are small, “conversation data importance 0.35” is evaluated. Based on importance scores, the detailed analysis module automatically applies rules such as “importance 0.8 or higher: detailed analysis (e.g., time-series decomposition, anomaly detection, graph generation), less than 0.5: summary analysis (e.g., average value, trend only)”. Examples of input to the AI include (1) one week's walking acceleration data (one-dimensional array), (2) one week's conversation text (100 utterances×7 days), (3) one week's meal events (structured data), (4) sleep data (sleep time, quality, number of turns), and (5) social media activity data (posting frequency, content). The AI output consists of (a) importance scores for each data type, (b) list of data for detailed analysis, and (c) list of data for summary analysis as structured data. For example, “walking data importance 0.92→detailed analysis”, “conversation data importance 0.35→summary analysis”. The analysis unit generates detailed analysis results as graphs or anomaly detection reports and presents summary analysis results as text summaries or average values only. As a subsequent process, the analysis unit sends analysis results to the alert unit or user presentation module and issues alerts or provides detailed explanations based on important data. As a technical effect, the analysis unit can realize optimal allocation of computational resources and efficient analysis by relying on AI-based data importance evaluation and automatic adjustment of detail level, rather than human heuristics or uniform analysis. This enables high-precision and efficient extraction of essential information for pre-dementia detection from vast data. Application fields include monitoring of elderly people at home, health management in nursing care facilities, remote medical support, and health management programs involving big data analysis. Furthermore, by optimizing parameters of importance evaluation algorithms for each user, it is possible to apply to personalized analysis and group optimization.

The analysis unit can apply different analysis algorithms according to the category of behavior data during analysis. The analysis unit defines categories of behavior data, for example. For example, the analysis unit defines categories based on the type of behavior data or classification of analysis targets. The analysis unit applies different analysis algorithms according to the category of behavior data, for example. For example, the analysis unit applies walking analysis algorithms to walking pattern data. The analysis unit applies natural language processing algorithms to conversation content data, for example. The analysis unit applies meal analysis algorithms to meal frequency data, for example. Thus, by applying appropriate analysis algorithms according to the category of behavior data, the analysis unit can improve analysis accuracy. Specifically, the analysis unit uses diverse behavior data received from the collection unit (e.g., time-series acceleration data of walking patterns, text data of conversation contents, structured data of meal events, sleep data, social media activity data) as input. The analysis unit implements a category classification module for each data type and automatically assigns category labels such as “walking”, “conversation”, “meal”, “sleep”, “social activity”. The analysis unit automatically selects the optimal AI algorithm for each category. For example, convolutional neural networks (CNN) or time-series anomaly detection algorithms (e.g., LSTM, autoregressive models) are applied to walking patterns to extract abnormalities in walking speed or rhythm. Natural language processing models (e.g., BERT, LSTM, Transformer) are applied to conversation contents to quantitatively evaluate vocabulary, semantic consistency, and utterance delay. Decision trees or rule-based meal analysis engines are applied to meal data to extract changes in meal count, nutritional balance, and calorie intake. Time-series analysis models (e.g., GRU, autoregressive models) are applied to sleep data to detect abnormalities in sleep time, quality, and number of turns. For social media activity, natural language processing and image analysis AI are combined to extract emotional tendencies in post contents and changes in activity frequency. Examples of input to the AI include (1) one day's walking acceleration data (one-dimensional array), (2) one week's conversation text (100 utterances×7 days), (3) one week's meal events (structured data), (4) sleep data (sleep time, quality, number of turns), and (5) social media post data (text, image features). The AI output consists of (a) anomaly detection scores for each category, (b) risk factor labels (e.g., “abnormal walking pattern”, “decline in conversation contents”, “poor meal balance”), and (c) recommended actions (e.g., “recommend visiting a medical institution”, “improve eating habits”) as structured data. The analysis unit integrates analysis results for each category and performs comprehensive pre-dementia risk assessment. As a subsequent process, the analysis unit sends analysis results to the alert unit or user presentation module and provides detailed explanations or issues alerts for each category. As a technical effect, the analysis unit can greatly improve analysis accuracy and efficiency by automatically selecting and applying the optimal AI model for each data type, rather than relying on human heuristics or uniform algorithm application. This enables high-precision and early detection of signs of pre-dementia from complex and diverse behavior data. Application fields include monitoring of elderly people at home, health management in nursing care facilities, remote medical support, and health management programs involving big data analysis. Furthermore, by optimizing parameters of category classification algorithms and analysis models for each user, it is possible to apply to personalized analysis and group optimization.

The analysis unit can estimate the user's emotions and adjust the length of analysis based on the estimated emotions. The analysis unit estimates the user's emotions, for example. For example, the analysis unit estimates the user's emotions using facial expression recognition technology. The analysis unit adjusts the length of analysis based on the user's emotions, for example. For example, if the user is relaxed, the analysis unit performs detailed analysis. If the user is feeling stressed, the analysis unit performs concise analysis. If the user is in a hurry, the analysis unit performs analysis focusing on key points. Thus, by adjusting the length of analysis according to the user's emotions, the analysis unit can provide appropriate analysis results for the user. Specifically, the analysis unit uses facial image data (e.g., 128×128 pixel RGB images, acquired at one-second intervals), audio data (e.g., one-second audio waveform sampled at 16 kHz), and biometric sensor data (e.g., heart rate, skin conductance as one-dimensional time-series data) as input for emotion estimation. The analysis unit inputs these data into multimodal AI models (e.g., combination of CNN and RNN, or Transformer-based emotion estimation models) to output emotion labels (e.g., “stress”, “relaxation”, “in a hurry”) and emotion intensity scores (continuous values from 0.0 to 1.0). For example, when facial images and audio waveforms are input simultaneously, the analysis unit outputs a probability distribution such as “stress: 0.80”, “relaxation: 0.10”, “in a hurry: 0.10”. Based on emotion estimation results, the analysis result length control module automatically applies rule-based output control such as “present only key points concisely when stress is high, present with detailed graphs and explanations when relaxed, present only key points in bullet points when in a hurry”. The analysis unit automatically adjusts the length of analysis results (e.g., number of characters in text summary, number of graphs, level of detail in explanations) according to the user's emotional state. Examples of AI output include (1) “stress state: present only key points (e.g., risk score 0.82, recommend visiting a medical institution)”, (2) “relaxation state: detailed analysis (e.g., graph of walking speed trends, changes in vocabulary in conversation contents, detailed explanation of meal patterns)”, and (3) “in a hurry: bullet point summary (e.g., risk factors, recommended actions only)”. As a subsequent process, after presenting analysis results, the analysis unit records the user's response (e.g., content confirmation, request for detailed display) and uses it for optimization of analysis length in future sessions. As a technical effect, the analysis unit can maximize understanding and utilization efficiency of analysis results without impairing user experience by relying on high-precision emotion estimation and automatic output control by AI, rather than subjective human judgment or uniform output. This enables reliable transmission of important information for pre-dementia detection while reducing user stress and burden. Application fields include monitoring of elderly people at home, health management in nursing care facilities, remote medical support, and health management programs involving stress management. Furthermore, by statistically analyzing the relationship between emotional state and analysis length for multiple users, it is possible to apply to group optimization and personalized health support.

The analysis unit can determine the priority of analysis based on the timing of collecting behavior data during analysis. The analysis unit evaluates the timing of collecting behavior data, for example. For example, the analysis unit evaluates the timing of collecting behavior data based on data freshness or collection timing. The analysis unit determines the priority of analysis based on the timing of collecting behavior data, for example. For example, the analysis unit prioritizes analysis of the latest behavior data. The analysis unit emphasizes the latest data while referring to past behavior data, for example. The analysis unit determines the priority of analysis based on the timing of collecting behavior data, for example. Thus, by determining the priority of analysis based on the timing of collecting behavior data, the analysis unit can prioritize analysis of the latest data. Specifically, the analysis unit uses timestamp information (e.g., UNIX epoch seconds, date and time labels) attached to behavior data received from the collection unit (e.g., walking patterns, conversation contents, meal events, sleep data, social media activities) as input. The analysis unit automatically calculates data freshness (e.g., difference from current time, elapsed time) and computes priority scores (continuous values from 0.0 to 1.0, with higher scores for newer data). Based on priority scores, the priority analysis module automatically applies rules such as “priority 0.8 or higher: immediate analysis, less than 0.5: deferred analysis”. Examples of input to the AI include (1) one day's walking acceleration data (with timestamp), (2) one week's conversation text (each utterance with recording time), (3) one week's meal events (with event occurrence time), (4) sleep data (with sleep and wake times), and (5) social media post data (with posting time). The AI output consists of (a) priority scores for analysis for each data type, (b) list of data for priority analysis, and (c) list of data for deferred analysis as structured data. For example, “walking data (today's data) priority 0.95→immediate analysis”, “conversation data (one week ago) priority 0.40→deferred analysis”. The analysis unit performs detailed analysis by AI models in order of priority analysis targets, and deferred targets are processed in batch or at low frequency. As a subsequent process, the analysis unit sends analysis results to the alert unit or user presentation module and issues alerts or explanations based on the latest data. As a technical effect, the analysis unit can achieve both real-time performance and analysis efficiency by relying on AI-based data freshness evaluation and automatic priority control, rather than human heuristics or uniform analysis. This enables rapid understanding of the latest behavioral changes for pre-dementia detection and realization of early intervention and appropriate response. Application fields include monitoring of elderly people at home, health management in nursing care facilities, remote medical support, and real-time health monitoring. Furthermore, by optimizing parameters of priority evaluation algorithms for each user, it is possible to apply to personalized analysis and group optimization.

The analysis unit can adjust the order of analysis based on the relevance of behavior data during analysis. The analysis unit evaluates the relevance of behavior data, for example. For example, the analysis unit evaluates the relevance of behavior data based on data correlation or relevance scoring. The analysis unit adjusts the order of analysis based on the relevance of behavior data, for example. For example, the analysis unit prioritizes analysis of highly relevant behavior data. The analysis unit defers analysis of behavior data with low relevance, for example. The analysis unit adjusts the order of analysis based on the relevance of behavior data, for example. Thus, by adjusting the order of analysis based on the relevance of behavior data, the analysis unit can perform efficient analysis. Specifically, the analysis unit uses multidimensional behavior data received from the collection unit (e.g., walking patterns, conversation contents, meal events, sleep data, social media activities) as input. The analysis unit calculates correlation coefficients (e.g., Pearson correlation, Spearman rank correlation) or covariance matrices between these data and computes relevance scores (continuous values from 0.0 to 1.0). Based on relevance scores, the analysis order determination module automatically applies rules such as “data pairs with relevance 0.8 or higher: simultaneous analysis, less than 0.5: deferred analysis”. Examples of input to the AI include (1) one week's walking acceleration data and sleep data (correlation analysis), (2) conversation contents and social media activities (commonality analysis of topics and emotional tendencies), and (3) meal events and health condition data (correlation analysis of nutrition intake and health changes). The AI output consists of (a) relevance scores for each data pair, (b) list of data pairs for priority analysis, and (c) list of data for deferred analysis as structured data. For example, “walking data and sleep data relevance 0.85→simultaneous analysis”, “conversation data and meal data relevance 0.30→deferred analysis”. The analysis unit prioritizes integrated analysis of highly relevant data and extracts complex risk factors or behavior change patterns. As a subsequent process, the analysis unit sends analysis results to the alert unit or user presentation module and issues alerts or explanations based on relevant data. As a technical effect, the analysis unit can realize discovery of complex behavior patterns and efficient analysis by relying on AI-based data relevance evaluation and automatic order control, rather than human heuristics or uniform analysis. This enables high-precision and efficient extraction of complex risk factors for pre-dementia detection. Application fields include monitoring of elderly people at home, health management in nursing care facilities, remote medical support, and health management programs involving big data analysis. Furthermore, by optimizing parameters of relevance evaluation algorithms for each user, it is possible to apply to personalized analysis and group optimization.

The alert unit can estimate the user's emotions and adjust the alert issuing method based on the estimated emotions. The alert unit estimates the user's emotions, for example. For example, the alert unit estimates the user's emotions using facial expression recognition technology. The alert unit adjusts the alert issuing method based on the user's emotions, for example. For example, if the user is relaxed, the alert unit issues a gentle alert. If the user is feeling stressed, the alert unit issues a concise and clear alert. If the user is in a hurry, the alert unit issues an alert that enables quick response. Thus, by adjusting the alert issuing method according to the user's emotions, the alert unit can issue appropriate alerts for the user. Specifically, the alert unit uses facial image data (e.g., 128×128 pixel RGB images, acquired at one-second intervals), audio data (e.g., one-second audio waveform sampled at 16 kHz), and biometric sensor data (e.g., heart rate, skin conductance as one-dimensional time-series data) as input for emotion estimation. The alert unit inputs these data into multimodal AI models (e.g., combination of CNN and RNN, or Transformer-based emotion estimation models) to output emotion labels (e.g., “stress”, “relaxation”, “in a hurry”) and emotion intensity scores (continuous values from 0.0 to 1.0). For example, when facial images and audio waveforms are input simultaneously, the alert unit outputs a probability distribution such as “stress: 0.75”, “relaxation: 0.15”, “in a hurry: 0.10”. Based on emotion estimation results, the alert issuing method determination module automatically applies rule-based output control such as “notify only key points concisely when stress is high, notify gently with detailed explanations when relaxed, notify quickly in bullet points when in a hurry”. The alert unit automatically selects notification means (e.g., smartphone notification, voice notification, email) and tone of notification text (e.g., polite, concise, emphasis on key points) according to emotional state. Examples of input to the AI include (1) facial image+audio waveform (simultaneously acquired), (2) time-series data of heart rate and skin conductance, and (3) combination data of multiple modalities. Examples of AI output include “stress: 0.80→concise notification”, “relaxation: 0.60→detailed notification”, “in a hurry: 0.70→key point notification”. After issuing an alert, the alert unit records the user's response (e.g., notification confirmation, level of understanding) and uses it for optimization of alert issuing method in future sessions. As a technical effect, the alert unit can maximize transmission efficiency and understanding of important information without impairing user experience by relying on high-precision emotion estimation and automatic notification control by AI, rather than subjective human judgment or uniform notification. This enables reliable and appropriate transmission of important alerts for pre-dementia detection while reducing user stress and burden. Application fields include monitoring of elderly people at home, health management in nursing care facilities, remote medical support, and health management programs involving stress management. Furthermore, by statistically analyzing the relationship between emotional state and notification method for multiple users, it is possible to apply to group optimization and personalized health support.

The alert unit can adjust the level of detail of alerts based on the importance of the prediction result when issuing an alert. For example, the alert unit evaluates the importance of the prediction result, such as by assessing the impact of the prediction result and the priority of the alert. The alert unit adjusts the level of detail of the alert according to the importance of the prediction result; for instance, it issues detailed alerts for important prediction results and concise alerts for less important ones. By adjusting the level of detail of alerts according to the importance of the prediction result, the alert unit can appropriately convey critical information. Specifically, the alert unit uses structured data received from the analysis unit, such as a pre-dementia risk score (continuous value from 0.0 to 1.0), risk factor labels (e.g., “abnormal walking patterns,” “decline in conversation contents”), and recommended actions (e.g., “recommend visiting a medical institution”) as input. The alert unit employs AI models (e.g., random forest, gradient boosting, neural networks with attention mechanisms) to calculate an importance score (0.0 to 1.0) for each prediction result. For example, a risk score of 0.9 or higher is evaluated as “importance 0.95,” while less than 0.5 is “importance 0.30.” Based on the importance score, the detail control module automatically applies rules such as “importance 0.8 or higher: detailed alert with explanation; less than 0.5: concise alert with key points only.” Examples of AI input include (1) risk score 0.92+factor “decreased walking speed”+recommendation “visit medical institution,” and (2) risk score 0.45+factor “decreased meal frequency”+recommendation “monitor progress.” AI output examples include “importance 0.92→detailed alert (with risk factors, recommended actions, and graphs)” and “importance 0.30→concise alert (key points only).” For detailed alerts, the alert unit provides risk trend graphs and explanations of factors; for concise alerts, only the risk score and recommended action are presented. After issuing an alert, the user's response (e.g., content confirmation, request for details) is recorded and used to optimize the level of detail for future alerts. As a technical effect, the alert unit achieves optimal allocation of computational resources and efficient notification by relying on AI-based importance evaluation and automatic detail control, rather than human heuristics or uniform notifications. This enables the transmission of essential information to users without excess or deficiency, facilitating prompt response and improved understanding. Application fields include monitoring of elderly people at home, health management in care facilities, remote medical support, and health management programs involving big data analysis. Furthermore, by optimizing the parameters of the importance evaluation algorithm for each user, personalized notifications and group optimization are also possible.

The alert unit can apply different alert issuing means according to the user's attribute information when issuing an alert. For example, the alert unit collects attribute information such as age, gender, and occupation. Based on the user's attribute information, the alert unit selects the optimal alert issuing means; for instance, prioritizing voice notifications for elderly users and using smartphone notification functions for younger users. By selecting the optimal alert issuing means according to the user's attribute information, the alert unit can issue alerts in a manner appropriate for each user. Specifically, the alert unit uses structured data such as age, gender, occupation, smartphone usage status, presence of hearing or visual impairment, and living arrangements as input. Based on this attribute information, the alert unit employs a notification means selection AI model (e.g., decision tree, rule-based engine) to automatically select the optimal notification means (e.g., voice notification, smartphone notification, email, proxy notification to family). For example, if an elderly user has low smartphone usage frequency and no hearing impairment, “voice notification+proxy notification to family” is prioritized; for a younger user with high smartphone usage, “smartphone notification” is selected. AI input examples include (1) age 75, low smartphone usage, no hearing impairment→voice notification; (2) age 35, high smartphone usage→smartphone notification; (3) age 80, visual impairment→voice notification+proxy notification to family. AI output examples are notification means labels such as “voice notification,” “smartphone notification,” “email notification,” and “proxy notification to family.” The alert unit records the notification means selection result and uses the user's response (e.g., notification confirmation rate, content comprehension) to optimize future notifications. As a technical effect, the alert unit achieves optimal information delivery for each user by relying on AI-based attribute information analysis and automatic notification means selection, rather than subjective human judgment or uniform notifications. This prevents missed notifications and misunderstandings, promoting prompt and reliable responses. Application fields include monitoring of elderly people at home, health management in care facilities, remote medical support, and personalized health management programs. Furthermore, by analyzing the relationship between attribute information and notification means at the group level, applications for group optimization and support for socially vulnerable populations are also possible.

The alert unit can estimate the user's emotions and determine the priority of alerts based on the estimated emotions. For example, the alert unit estimates the user's emotions using facial recognition technology. Based on the user's emotions, the alert unit determines the priority of alerts; for instance, prioritizing important alerts when the user is relaxed, prioritizing concise alerts when the user is stressed, and prioritizing alerts that enable rapid response when the user is in a hurry. By determining the priority of alerts according to the user's emotions, the alert unit can issue important alerts preferentially. Specifically, the alert unit uses facial image data (128×128 pixel RGB images), audio data (16 kHz sampled audio waveform), and biometric sensor data (heart rate, skin conductance) as input, and outputs emotion labels (“stress,” “relaxed,” “in a hurry”) and emotion intensity scores (0.0 to 1.0) using a multimodal AI model (CNN+RNN or Transformer-based). The alert unit inputs the emotion estimation result and alert content (e.g., risk score, factor label, recommended action) into a priority determination module, which automatically applies rules such as “when stress is high, prioritize important alerts with key points only; when relaxed, prioritize detailed alerts; when in a hurry, prioritize immediate response alerts.” AI input examples include (1) emotion score “stress 0.85”+multiple alert candidates, and (2) emotion score “relaxed 0.70”+multiple alert candidates. AI output examples are priority labels such as “important alert priority,” “concise alert priority,” and “immediate alert priority.” The alert unit records the prioritization result and uses the user's response (e.g., notification confirmation, response speed) to optimize future prioritization. As a technical effect, the alert unit maximizes the efficiency of important information transmission and response speed without impairing the user experience, by relying on AI-based emotion estimation and automatic priority control rather than subjective human judgment or uniform notifications. This reduces user stress and burden while ensuring that important alerts for pre-dementia detection are transmitted in the correct order. Application fields include monitoring of elderly people at home, health management in care facilities, remote medical support, and health management programs involving stress management. Furthermore, by statistically analyzing the relationship between the emotional states of multiple users and alert priorities, applications for group optimization and personalized health support are also possible.

The alert unit can select the optimal alert issuing means by considering the user's geographic location information when issuing an alert. For example, the alert unit collects the user's geographic location information using GPS data or location information services. Based on the user's geographic location information, the alert unit selects the optimal alert issuing means; for instance, using smartphone notification functions when the user is at home, and prioritizing voice notifications when the user is outside. By selecting the optimal alert issuing means according to the user's geographic location information, the alert unit can issue alerts in a manner appropriate for each user. Specifically, the alert unit uses GPS data (latitude, longitude, altitude, timestamped time-series data for 24 hours at 1-minute intervals) and Wi-Fi/Bluetooth beacon information obtained from the user's smartphone or wearable device as input. The alert unit matches this location information with a map database and automatically assigns location labels such as “home,” “workplace,” “park,” and “medical institution.” For each location label, the alert unit implements a rule-based engine that applies notification means selection rules (e.g., smartphone notification at home, voice notification when outside, proxy notification to family at medical institutions). AI input examples include (1) GPS data+location label “home,” (2) GPS data+location label “outside,” and (3) Wi-Fi beacon+location label “medical institution.” AI output examples are notification means labels such as “smartphone notification,” “voice notification,” and “proxy notification to family.” The alert unit records the notification means selection result and uses the user's response (e.g., notification confirmation rate, content comprehension) to optimize future notifications. As a technical effect, the alert unit achieves optimal information delivery according to the user's situation by relying on AI-based location information analysis and automatic notification means selection, rather than subjective human judgment or uniform notifications. This prevents missed notifications and misunderstandings, promoting prompt and reliable responses. Application fields include monitoring of elderly people at home, health management during outings, behavior monitoring in care facilities, and regional medical collaboration. Furthermore, by analyzing the relationship between location information and notification means at the group level, applications for optimal notification strategies at the regional level and public health policy are also possible.

The alert unit can analyze the user's social media activities and adjust the content of alerts when issuing an alert. For example, the alert unit analyzes the user's social media activities, such as analyzing post content and activity frequency. Based on the analysis of the user's social media activities, the alert unit issues relevant alert content, adjusting the content according to information shared by the user on social media. By analyzing the user's social media activities, the alert unit can issue highly relevant alert content. Specifically, the alert unit automatically acquires public post data (text content, posting time, posting frequency, image metadata, video playback count, and other structured data) via API from multiple social media platforms used by the user. The acquired post data is input into a natural language processing engine and image analysis AI model to extract emotional tendencies of posts (e.g., “positive,” “negative,” “neutral”), topic classification (e.g., “health,” “hobbies,” “family”), and patterns of activity frequency changes. AI input examples include (1) one week of text posts (content, posting time, emotion score), (2) image post metadata (image feature vectors, posting time), and (3) time-series data of posting frequency (daily post count vector). AI output includes (a) social activity score (0.0 to 1.0), (b) activity tendency label (e.g., “decreased activity,” “increased health topics,” “tendency toward isolation”), and (c) alert content adjustment instructions (e.g., “recommend going out,” “recommend increasing conversation”). Based on these AI outputs, the alert content generation module automatically applies rules such as “recommend going out or socializing when activity decreases, provide health maintenance advice when health topics increase, and provide guidance on family or community support when isolation is detected.” The alert unit records the alert content adjustment result and uses the user's response (e.g., notification confirmation, behavioral change) to optimize future alerts. As a technical effect, the alert unit achieves optimal information delivery according to the user's social situation by relying on AI-based social activity analysis and automatic content adjustment, rather than human observation or uniform notifications. This promotes early detection of social isolation and decreased activity, enabling appropriate intervention. Application fields include monitoring of elderly people at home, social activity monitoring in care facilities, remote medical support, health management programs, and creation of isolation risk maps at the regional level. Furthermore, by analyzing the relationship between social activity and alert content at the group level, applications for public health policy and community support strategies are also possible.

The system according to the embodiment is not limited to the examples described above and can be variously modified as follows. Specifically, the system can switch the architecture of the AI model to various configurations such as convolutional neural networks, recurrent neural networks, Transformer-based models, decision trees, and gradient boosting. The data collection targets can also be expanded to include walking patterns, conversation contents, meal events, sleep data, social media activities, physiological data (heart rate, blood pressure, body temperature), and environmental sensor data (room temperature, humidity, illuminance). Furthermore, the input data formats for AI can accommodate various variations, including one-dimensional time-series arrays, two-dimensional image tensors, structured tables, natural language text, and multimodal data. The AI output can also support various formats, such as risk scores, anomaly detection labels, recommended actions, detailed analysis reports, graphs, infographics, and voice explanations. Subsequent processing may include alert issuance, optimization of notification means, content, and timing according to user attributes, emotions, location information, and social activities, recording user responses and reflecting them in AI training data, and creating risk maps or applying to public health policies at the group level. As a technical effect, the system achieves comprehensive improvement in analysis accuracy, notification efficiency, user experience, and social value by combining multiple data, multiple AI models, diverse outputs, and flexible subsequent processing, thereby overcoming dependence on single data and single models in conventional systems. Application fields include monitoring of elderly people at home, health management in care facilities, remote medical support, health management programs, creation of health risk maps at the regional level, public health policy, and personalized medical and care support.

The collection unit can collect physiological data of the user in addition to behavior data. For example, by collecting physiological data such as heart rate, blood pressure, and body temperature, the user's health condition can be understood in greater detail. The analysis unit analyzes the collected physiological data and predicts the possibility that the user corresponds to a pre-dementia state by combining it with behavior data. For example, by analyzing fluctuations in heart rate, changes in blood pressure, and abnormalities in body temperature, signs of dementia can be detected. The alert unit can issue alerts regarding the user's health condition based on the analysis result. For example, if the heart rate is abnormally high or blood pressure fluctuates rapidly, the alert unit can issue an alert recommending a visit to a medical institution. This enables the user to take early measures not only for dementia but also for other health issues. Specifically, the collection unit automatically acquires physiological data such as heart rate (e.g., time-series data at 1-minute intervals), blood pressure (e.g., measurements twice daily, morning and evening), and body temperature (e.g., four measurements per day) from wearable devices or home biometric sensors and stores them in a database as time-series data. The analysis unit inputs these physiological data into time-series analysis models (e.g., LSTM, autoregressive models) and anomaly detection algorithms (e.g., Isolation Forest, change point detection) to automatically extract sudden fluctuations in heart rate, abnormal blood pressure patterns, and sustained increases or decreases in body temperature. AI input examples include (1) one week of heart rate time-series data (1-minute intervals), (2) one month of blood pressure measurements (morning and evening), and (3) one week of body temperature measurements (four times per day). AI output includes (a) physiological data anomaly score (0.0 to 1.0), (b) anomaly factor label (e.g., “heart rate fluctuation,” “sudden blood pressure change,” “body temperature abnormality”), and (c) recommended action (e.g., “recommend visiting a medical institution,” “monitor progress”). Based on these outputs, the alert unit issues alerts to the user or family, such as “heart rate is abnormally high, recommend visiting a medical institution” or “blood pressure has fluctuated rapidly, caution is required.” Subsequent processing includes recording the user's response (e.g., whether a visit to a medical institution was made, whether information was shared with family) after alert issuance and using it as AI training data for future analysis. As a technical effect, the collection unit, analysis unit, and alert unit achieve early detection and response to various health risks, not limited to pre-dementia, by relying on AI-based high-frequency, high-precision physiological data analysis and automatic alert issuance, rather than human observation or manual recording. This greatly contributes to maintaining user health, preventing severe conditions, and reducing medical burden. Application fields include monitoring of elderly people at home, chronic disease management, remote medical support, health management programs, and personalized medicine. Furthermore, by aggregating anonymized physiological data from multiple users, applications for creating health risk maps at the group level and public health policy are also possible.

The analysis unit can detect changes in the user's social activities based on behavior data. For example, if the user's frequency of going out or interacting with friends decreases compared to before, signs of social isolation can be detected. The analysis unit evaluates these changes as signs of dementia and reflects them in the prediction result. The alert unit can issue alerts encouraging the user to increase social activities when signs of social isolation are detected. For example, the alert unit can issue alerts recommending participation in local events or community activities. This enables the user to prevent social isolation and reduce the risk of dementia. Specifically, the analysis unit uses multidimensional behavior data received from the collection unit (e.g., time-series data of outing frequency, frequency of conversations with friends, frequency of social media posts, event participation history) as input. These data are input into time-series analysis models (e.g., LSTM, autoregressive models) and clustering algorithms (e.g., k-means, DBSCAN) to automatically extract patterns such as decreasing frequency of outings, declining frequency of interactions, and reduced posting activity. AI input examples include (1) one month of outing frequency (daily numerical vector), (2) one week of conversation event counts (with event time and partner information), and (3) one month of social media post counts (daily vector). AI output includes (a) social activity score (continuous value from 0.0 to 1.0, e.g., 0.20 indicates decreased activity), (b) isolation tendency label (e.g., “decreased activity,” “reduced interaction”), and (c) recommended action (e.g., “recommend going out,” “recommend increasing interaction”). For example, if the number of outings is halved in one month and the number of conversation events also decreases, the analysis unit assigns a “decreased activity” label and increases the risk score. Based on these outputs, the alert unit issues alerts to the user or family, such as “Recently, outings and interactions have decreased. Participation in local events or community activities is recommended.” Subsequent processing includes recording the user's response (e.g., whether events were attended, whether interaction increased) after alert issuance and using it as AI training data for future analysis. As a technical effect, the analysis unit achieves high-precision and early detection of signs of social isolation by combining AI-based high-dimensional feature extraction and multifaceted analysis, rather than relying on human observation or simple count tracking. This enables objective understanding of social isolation, a risk factor for pre-dementia, based on data and facilitates appropriate data collection and intervention. Application fields include monitoring of elderly people at home, social activity monitoring in care facilities, remote medical support, health management programs, and creation of isolation risk maps at the regional level. Furthermore, by aggregating anonymized social activity data from multiple users, applications for understanding group social activity tendencies and public health policy are also possible.

The collection unit can collect sleep data of the user in addition to behavior data. For example, by collecting data such as sleep duration, sleep quality, and number of turnovers, the user's sleep state can be understood in detail. The analysis unit analyzes the collected sleep data and predicts the possibility that the user corresponds to a pre-dementia state by combining it with behavior data. For example, by analyzing decreased sleep duration, reduced sleep quality, and increased number of turnovers, signs of dementia can be detected. The alert unit can issue alerts regarding the user's sleep state based on the analysis result. For example, if sleep duration is short or sleep quality is reduced, the alert unit can issue alerts recommending improvement of the sleep environment. This enables the user to improve sleep state and reduce the risk of dementia. Specifically, the collection unit automatically acquires time-series data such as sleep duration (e.g., daily bedtime and wake-up time), sleep quality (e.g., ratio of deep sleep to light sleep, sleep efficiency), and number of turnovers (e.g., number per night) from wearable devices or bed sensors and stores them in a database. The analysis unit inputs these data into time-series analysis models (e.g., LSTM, autoregressive models) and anomaly detection algorithms (e.g., Isolation Forest, change point detection) to automatically extract abnormal patterns such as decreasing sleep duration, reduced sleep efficiency, and increased number of turnovers. AI input examples include (1) one week of sleep duration data (daily numerical vector), (2) one week of sleep efficiency data (time-series of deep sleep ratio), and (3) one week of turnover counts (number per night). AI output includes (a) sleep anomaly score (0.0 to 1.0), (b) anomaly factor label (e.g., “decreased sleep duration,” “reduced sleep efficiency,” “increased turnovers”), and (c) recommended action (e.g., “recommend improving sleep environment,” “recommend visiting a medical institution”). For example, if sleep duration drops below an average of five hours per night for a week and the number of turnovers increases, the analysis unit assigns a “sleep anomaly” label and increases the risk score. Based on these outputs, the alert unit issues alerts to the user or family, such as “Recently, sleep duration has decreased. Review of sleep environment or visiting a medical institution is recommended.” Subsequent processing includes recording the user's response (e.g., whether sleep environment was improved, whether a visit to a medical institution was made) after alert issuance and using it as AI training data for future analysis. As a technical effect, the collection unit, analysis unit, and alert unit achieve early detection and response to various health risks, not limited to pre-dementia, by relying on AI-based high-frequency, high-precision sleep data analysis and automatic alert issuance, rather than human observation or manual recording. This greatly contributes to maintaining user health, preventing severe conditions, and reducing medical burden. Application fields include monitoring of elderly people at home, sleep disorder management, remote medical support, health management programs, and personalized medicine. Furthermore, by aggregating anonymized sleep data from multiple users, applications for creating health risk maps at the group level and public health policy are also possible.

The analysis unit can detect changes in the user's cognitive function based on behavior data. For example, by analyzing the completion time and frequency of mistakes in daily tasks performed by the user, declines in cognitive function can be detected. The analysis unit evaluates these changes as signs of dementia and reflects them in the prediction result. The alert unit can issue alerts recommending training or activities to maintain cognitive function when a decline is detected. For example, the alert unit can issue alerts recommending the use of brain training apps, reading, or puzzles to stimulate cognitive function. This enables the user to maintain cognitive function and reduce the risk of dementia. Specifically, the analysis unit uses daily task execution data received from the collection unit (e.g., shopping list completion time, medication management app operation history, records of household task execution, error occurrence count) as input. These data are input into time-series analysis models (e.g., LSTM, autoregressive models) and anomaly detection algorithms (e.g., Isolation Forest, change point detection) to automatically extract abnormal patterns such as delayed task completion time, increased frequency of mistakes, and confusion in operation procedures. AI input examples include (1) one week of task completion times (vector of required times for each task), (2) one month of error occurrence counts (daily values), and (3) app operation history (time-series data of operation procedures). AI output includes (a) cognitive function decline score (0.0 to 1.0), (b) anomaly factor label (e.g., “task delay,” “increased mistakes,” “operation confusion”), and (c) recommended action (e.g., “recommend brain training,” “recommend reading,” “recommend puzzles”). For example, if task completion time is delayed by an average of 30% over a week and error occurrence count increases, the analysis unit assigns a “cognitive function decline” label and increases the risk score. Based on these outputs, the alert unit issues alerts to the user or family, such as “Recently, task completion is taking longer. Brain training, reading, or puzzle activities are recommended.” Subsequent processing includes recording the user's response (e.g., whether training was performed, record of activities) after alert issuance and using it as AI training data for future analysis. As a technical effect, the analysis unit achieves high-precision and early detection of signs of cognitive function decline by combining AI-based high-dimensional feature extraction and multifaceted analysis, rather than relying on human observation or simple recording. This enables objective understanding of cognitive function decline, a risk factor for pre-dementia, based on data and facilitates appropriate data collection and intervention. Application fields include monitoring of elderly people at home, cognitive function monitoring in care facilities, remote medical support, health management programs, and personalized cognitive function support. Furthermore, by aggregating anonymized cognitive function data from multiple users, applications for understanding group cognitive function tendencies and public health policy are also possible.

The collection unit can collect dietary data of the user in addition to behavior data. For example, by collecting data such as meal contents, calorie intake, and nutritional balance, the user's dietary habits can be understood in detail. The analysis unit analyzes the collected dietary data and predicts the possibility that the user corresponds to a pre-dementia state by combining it with behavior data. For example, by analyzing imbalances in nutritional balance and excess or deficiency in calorie intake, signs of dementia can be detected. The alert unit can issue alerts regarding the user's dietary habits based on the analysis result. For example, if nutritional balance is skewed or calorie intake is excessive or insufficient, the alert unit can issue alerts recommending improvement of dietary habits. This enables the user to improve dietary habits and reduce the risk of dementia. Specifically, the collection unit automatically acquires structured data such as meal contents (e.g., types of main dish, side dish, staple food), calorie intake (e.g., calorie value per meal), and nutritional balance (e.g., intake amounts of protein, fat, carbohydrates, vitamins, minerals) from meal recording apps or wearable devices and stores them in a database as time-series data. The analysis unit inputs these data into decision trees, rule-based dietary analysis engines, and time-series analysis models (e.g., LSTM) to automatically extract imbalances in nutritional balance, excess or deficiency in calorie intake, and changes in dietary patterns. AI input examples include (1) one week of meal content data (vector of items, calories, nutrients for each meal), (2) one month of calorie intake (daily values), and (3) one week of nutritional balance data (vector of intake amounts for each nutrient). AI output includes (a) dietary anomaly score (0.0 to 1.0), (b) anomaly factor label (e.g., “nutritional imbalance,” “excess calories,” “insufficient calories”), and (c) recommended action (e.g., “recommend improving dietary habits,” “recommend consulting a nutritionist”). For example, if protein intake is less than half the recommended value for a week and calorie intake is excessive, the analysis unit assigns a “nutritional imbalance” label and increases the risk score. Based on these outputs, the alert unit issues alerts to the user or family, such as “Recently, nutritional balance is skewed. Review of dietary habits or consulting a nutritionist is recommended.” Subsequent processing includes recording the user's response (e.g., whether dietary habits were improved, whether a nutritionist was consulted) after alert issuance and using it as AI training data for future analysis. As a technical effect, the collection unit, analysis unit, and alert unit achieve early detection and response to various health risks, not limited to pre-dementia, by relying on AI-based high-frequency, high-precision dietary data analysis and automatic alert issuance, rather than human observation or manual recording. This greatly contributes to maintaining user health, preventing severe conditions, and reducing medical burden. Application fields include monitoring of elderly people at home, nutrition management, remote medical support, health management programs, and personalized medicine. Furthermore, by aggregating anonymized dietary data from multiple users, applications for creating health risk maps at the group level and public health policy are also possible.

The analysis unit can estimate the user's emotions and adjust the presentation method of analysis results based on the estimated emotions. For example, if the user is stressed, the analysis results can be summarized concisely; if the user is relaxed, detailed analysis results can be provided; and if the user is in a hurry, the analysis results can be presented with key points only. This enables the user to receive appropriate information according to their emotional state and utilize the analysis results more effectively. Specifically, the analysis unit uses facial image data (e.g., 128×128 pixel RGB images acquired at 1-second intervals), audio data (e.g., 1-second audio waveform sampled at 16 kHz), and biometric sensor data (e.g., one-dimensional time-series data of heart rate and skin conductance) as input for emotion estimation. These data are input into a multimodal AI model (e.g., a combination of CNN+RNN or a Transformer-based emotion estimation model), which outputs emotion labels (e.g., “stress,” “relaxed,” “in a hurry”) and emotion intensity scores (continuous values from 0.0 to 1.0). For example, when facial images and audio waveforms are input simultaneously, the analysis unit outputs a probability distribution such as “stress: 0.72,” “relaxed: 0.18,” “in a hurry: 0.10.” Based on the emotion estimation result, the analysis result presentation method determination module automatically applies rule-based output control such as “when stress is high, present only key points concisely; when relaxed, present detailed graphs and explanations; when in a hurry, present only key points in bullet points.” The analysis unit automatically selects the presentation format of analysis results (e.g., text summary, detailed graphs, infographics, voice explanations) according to the user's emotional state. AI output examples include (1) “stress state: present only key points (e.g., risk score 0.82, recommend visiting a medical institution),” (2) “relaxed state: detailed analysis (e.g., walking speed trend graph, vocabulary change in conversation contents, detailed explanation of dietary patterns),” and (3) “in a hurry: bullet point summary (e.g., risk factors and recommended actions only).” Subsequent processing includes recording the user's response (e.g., content confirmation, request for details) after presenting the analysis results and using it to optimize future analysis presentation. As a technical effect, the analysis unit maximizes the user's understanding and utilization efficiency of analysis results without impairing the user experience, by relying on AI-based high-precision emotion estimation and automatic output control, rather than subjective human judgment or uniform output. This reduces user stress and burden while ensuring that important information for pre-dementia detection is reliably transmitted. Application fields include monitoring of elderly people at home, health management in care facilities, remote medical support, and health management programs involving stress management. Furthermore, by statistically analyzing the relationship between the emotional states of multiple users and analysis presentation, applications for group optimization and personalized health support are also possible.

The alert unit can estimate the user's emotions and adjust the content of alerts based on the estimated emotions. For example, if the user is stressed, the alert content can be made concise, providing only the necessary information; if the user is relaxed, detailed alert content can be provided; and if the user is in a hurry, alerts can be issued with key points for rapid response. This enables the user to receive appropriate alerts according to their emotional state and respond quickly and effectively. Specifically, the alert unit uses facial image data (e.g., 128×128 pixel RGB images acquired at 1-second intervals), audio data (e.g., 1-second audio waveform sampled at 16kHz), and biometric sensor data (e.g., one-dimensional time-series data of heart rate and skin conductance) as input for emotion estimation. These data are input into a multimodal AI model (e.g., a combination of CNN+RNN or a Transformer-based emotion estimation model), which outputs emotion labels (e.g., “stress,” “relaxed,” “in a hurry”) and emotion intensity scores (continuous values from 0.0 to 1.0). For example, when facial images and audio waveforms are input simultaneously, the alert unit outputs a probability distribution such as “stress: 0.75,” “relaxed: 0.15,” “in a hurry: 0.10.” Based on the emotion estimation result, the alert content generation module automatically applies rule-based output control such as “when stress is high, notify only key points concisely; when relaxed, notify with detailed explanations; when in a hurry, notify only key points in bullet points.” The alert unit automatically selects the tone of the notification (e.g., polite, concise, key point emphasis) and the level of detail (e.g., with graphs, key points only) according to the user's emotional state. AI output examples include “stress state: concise notification (e.g., risk score 0.82, recommend visiting a medical institution),” “relaxed state: detailed notification (e.g., risk factors, recommended actions, with graphs),” and “in a hurry: key point notification (e.g., recommended actions only).” After issuing an alert, the user's response (e.g., notification confirmation, content comprehension) is recorded and used to optimize future alert content. As a technical effect, the alert unit maximizes the efficiency and comprehension of important information transmission without impairing the user experience, by relying on AI-based high-precision emotion estimation and automatic notification content control, rather than subjective human judgment or uniform notifications. This reduces user stress and burden while ensuring that important alerts for pre-dementia detection are reliably and appropriately transmitted. Application fields include monitoring of elderly people at home, health management in care facilities, remote medical support, and health management programs involving stress management. Furthermore, by statistically analyzing the relationship between the emotional states of multiple users and notification content, applications for group optimization and personalized health support are also possible.

The collection unit can estimate the user's emotions and adjust the frequency of data collection based on the estimated emotions. For example, if the user is stressed, the frequency of data collection can be reduced to lessen the user's burden; if the user is relaxed, the frequency can be increased to collect more detailed data; and if the user is in a hurry, only important data can be prioritized for collection. This enables the user to receive appropriate data collection according to their emotional state, reducing burden while ensuring necessary data is obtained. Specifically, the collection unit uses facial image data (e.g., 128×128 pixel RGB images acquired at 1-second intervals), audio data (e.g., 1-second audio waveform sampled at 16 kHz), and biometric sensor data (e.g., one-dimensional time-series data of heart rate and skin conductance) as input for emotion estimation. These data are input into convolutional neural networks (CNN), recurrent neural networks (RNN), or multimodal Transformer models, which output emotion classification labels (e.g., “stress,” “relaxed,” “in a hurry”) and emotion intensity scores (continuous values from 0.0 to 1.0). For example, when facial images and audio waveforms are input simultaneously, the collection unit outputs a probability distribution such as “stress: 0.78,” “relaxed: 0.12,” “in a hurry: 0.10.” Based on these output values, the collection timing control module automatically applies rule-based scheduling such as “when stress is high, collect at 10-minute intervals; when relaxed, at 1-minute intervals; when in a hurry, collect only important data immediately.” The collection unit monitors emotion estimation results in real time and automatically reduces collection frequency when a threshold (e.g., stress score 0.7 or higher) is exceeded. To minimize user burden while ensuring necessary data, the collection unit can also implement an optimal collection timing algorithm (e.g., automatic parameter adjustment by reinforcement learning) according to emotional state. As a technical effect, the collection unit maximizes data collection efficiency without impairing the user experience, by relying on AI-based high-precision emotion estimation and automatic scheduling, rather than subjective human judgment or manual settings. This enables stable acquisition of high-quality behavior data for pre-dementia detection while greatly reducing user stress and burden. Application fields include monitoring of elderly people at home, health management in care facilities, remote medical support, and health management programs involving stress management. Furthermore, by statistically analyzing the relationship between the emotional states of multiple users and collection frequency, applications for group optimization and personalized health support are also possible.

The analysis unit can estimate the user's emotions and determine the priority of analysis based on the estimated emotions. For example, if the user is stressed, only important data can be prioritized for analysis and results provided quickly; if the user is relaxed, detailed analysis can be performed and comprehensive results provided; and if the user is in a hurry, analysis can be focused on key points for rapid response. This enables the user to receive appropriate analysis results according to their emotional state and utilize them effectively. Specifically, the analysis unit uses facial image data (e.g., 128×128 pixel RGB images acquired at 1-second intervals), audio data (e.g., 1-second audio waveform sampled at 16 kHz), and biometric sensor data (e.g., one-dimensional time-series data of heart rate and skin conductance) as input for emotion estimation. These data are input into a multimodal AI model (e.g., a combination of CNN+RNN or a Transformer-based emotion estimation model), which outputs emotion labels (e.g., “stress,” “relaxed,” “in a hurry”) and emotion intensity scores (continuous values from 0.0 to 1.0). For example, when facial images and audio waveforms are input simultaneously, the analysis unit outputs a probability distribution such as “stress: 0.80,” “relaxed: 0.10,” “in a hurry: 0.10.” Based on the emotion estimation result, the analysis priority determination module automatically applies rule-based output control such as “when stress is high, prioritize analysis of important data; when relaxed, perform detailed analysis of all data; when in a hurry, analyze only key points.” The analysis unit automatically sets the priority of target data (e.g., walking patterns, conversation contents, meal events, sleep data) according to emotional state, achieving optimal allocation of computational resources and efficient analysis. AI output examples include “stress state: prioritize analysis of important data,” “relaxed state: detailed analysis of all data,” and “in a hurry: key point analysis.” Subsequent processing includes sending analysis results to the alert unit or user presentation module, recording the user's response (e.g., content confirmation, request for details), and using it to optimize future analysis priority. As a technical effect, the analysis unit maximizes the user's understanding and utilization efficiency of analysis results without impairing the user experience, by relying on AI-based high-precision emotion estimation and automatic priority control, rather than subjective human judgment or uniform analysis. This reduces user stress and burden while ensuring that important information for pre-dementia detection is reliably transmitted. Application fields include monitoring of elderly people at home, health management in care facilities, remote medical support, and health management programs involving stress management. Furthermore, by statistically analyzing the relationship between the emotional states of multiple users and analysis priority, applications for group optimization and personalized health support are also possible.

The alert unit can estimate the user's emotions and adjust the timing of alert issuance based on the estimated emotions. For example, if the user is stressed, alert issuance can be delayed and issued when the user is relaxed; if the user is relaxed, alerts can be issued immediately to promote prompt response; and if the user is in a hurry, only important alerts can be issued immediately for rapid response. This enables the user to receive alerts at appropriate timing according to their emotional state and respond effectively. Specifically, the alert unit uses facial image data (e.g., 128×128 pixel RGB images acquired at 1-second intervals), audio data (e.g., 1-second audio waveform sampled at 16 kHz), and biometric sensor data (e.g., one-dimensional time-series data of heart rate and skin conductance) as input for emotion estimation. These data are input into a multimodal AI model (e.g., a combination of CNN+RNN or a Transformer-based emotion estimation model), which outputs emotion labels (e.g., “stress,” “relaxed,” “in a hurry”) and emotion intensity scores (continuous values from 0.0 to 1.0). For example, when facial images and audio waveforms are input simultaneously, the alert unit outputs a probability distribution such as “stress: 0.80,” “relaxed: 0.10,” “in a hurry: 0.10.” Based on the emotion estimation result, the alert issuance timing control module automatically applies rule-based output control such as “when stress is high, delay issuance; when relaxed, issue immediately; when in a hurry, issue only important alerts immediately.” The alert unit implements an optimal timing algorithm (e.g., automatic parameter adjustment by reinforcement learning) and uses the user's response (e.g., notification confirmation rate, response speed) to optimize future timing. AI output examples include “stress state: delayed issuance,” “relaxed state: immediate issuance,” and “in a hurry: immediate issuance of important alerts.” As a technical effect, the alert unit maximizes the efficiency and speed of important information transmission without impairing the user experience, by relying on AI-based high-precision emotion estimation and automatic timing control, rather than subjective human judgment or uniform notifications. This reduces user stress and burden while ensuring that important alerts for pre-dementia detection are reliably and appropriately transmitted at the correct timing. Application fields include monitoring of elderly people at home, health management in care facilities, remote medical support, and health management programs involving stress management. Furthermore, by statistically analyzing the relationship between the emotional states of multiple users and issuance timing, applications for group optimization and personalized health support are also possible.

The following is a brief explanation of the processing flow of Example of the Embodiment. Specifically, in this system, the collection unit automatically acquires daily activity data of the user (e.g., time-series acceleration data of walking patterns, text data of conversation contents, structured data of meal events, sleep data, social media activity data, physiological data, etc.) from various sensors and applications and stores them in a database as time-series data. The collection unit automatically detects missing or abnormal data and performs preprocessing such as outlier removal and normalization. Furthermore, the collection unit simultaneously collects various attribute information such as emotion estimation of the user (e.g., inputting facial images, audio, and biometric sensor data into an AI model to output emotion labels and intensity scores), health condition, living environment, geographic location information, and social activities. The analysis unit inputs the multidimensional data received from the collection unit into AI architectures such as convolutional neural networks (CNN), recurrent neural networks (RNN), Transformer-based models, decision trees, and gradient boosting, and automatically extracts abnormalities in walking patterns, changes in conversation contents, anomalies in meal, sleep, and physiological data, declines in social activities, and changes in cognitive function. Based on the AI model output (e.g., risk score, anomaly factor label, recommended action, detailed analysis report), the analysis unit performs threshold judgment and branching processing, and uses the results for notification to the alert unit and accumulation of subsequent training data. The alert unit automatically selects the optimal notification means, content, and timing according to the prediction result received from the analysis unit and the user's attributes, emotions, location information, and social activities, and issues alerts to the user or family. After issuance, the alert unit records the user's response (e.g., notification confirmation, whether a visit to a medical institution was made, behavioral change) and uses it as AI training data for future analysis. As a technical effect, the system achieves early detection and response to various health risks, not limited to pre-dementia, by relying on AI-based high-dimensional data analysis and automatic alert issuance, rather than human observation or manual recording. This greatly contributes to maintaining user health, preventing severe conditions, and reducing medical burden. Application fields include monitoring of elderly people at home, health management in care facilities, remote medical support, health management programs, and personalized medicine. Furthermore, by aggregating anonymized data from multiple users, applications for creating health risk maps at the group level and public health policy are also possible.

Step 1: The collection unit collects daily activity data of the user. The daily activity data of the user includes, for example, walking patterns, conversation contents, and meal frequency. The collection unit collects walking patterns using pedometers and acceleration sensors, conversation contents using speech recognition technology, and meal frequency using meal recording apps. Step 2: The analysis unit analyzes the data collected by the collection unit and predicts the possibility that the user corresponds to a pre-dementia state. The analysis unit analyzes the collected data using AI, analyzes the user's behavior patterns, and detects signs of dementia, such as changes in walking patterns, conversation contents, and meal frequency. Step 3: The alert unit issues alerts based on the prediction result obtained by the analysis unit. The alert unit issues alerts using smartphone notification functions, email, voice notifications, etc., including content recommending early visits to medical institutions if the user may correspond to a pre-dementia state. Specifically, in Step 1, the collection unit automatically acquires multidimensional data such as daily step count from pedometers and acceleration sensors (e.g., 10,000 steps per day), walking speed (e.g., 1.2 m/s), time-series acceleration data of walking rhythm (e.g., 50 Hz sampling, about 4.32 million samples per day), speech texts converted by speech recognition engines (e.g., 100 sentences per day), and meal events from meal recording apps (e.g., three times per day, each event with meal contents, time, and calorie information), and stores them in a database as time-series data. In Step 2, the analysis unit inputs these data into AI architectures such as convolutional neural networks (CNN), recurrent neural networks (RNN), and Transformer-based time-series analysis models to automatically extract abnormalities in walking patterns (e.g., decreased walking speed, disrupted rhythm), changes in conversation contents (e.g., decreased vocabulary, delayed speech, reduced semantic consistency), and changes in meal frequency (e.g., decreased meal count, irregular meal times). AI input examples include one day of walking acceleration data (50 Hz×86,400 seconds=4.32 million points in a one-dimensional array), one day of speech text (e.g., “The weather is nice today,” “I ate rice,” etc., 100 sentences), and one week of meal events (structured data with meal contents, calories, and time for each event). AI output includes pre-dementia risk score (continuous value from 0.0 to 1.0), risk factor label (e.g., “abnormal walking patterns,” “decline in conversation contents”), and recommended action (e.g., “recommend visiting a medical institution,” “monitor progress”). In Step 3, the alert unit receives these outputs and issues specific alerts to the user or family using smartphone notification functions, email, and voice notification APIs, such as “There is a high possibility of pre-dementia; please visit a medical institution as soon as possible.” Subsequent processing includes recording the user's response (e.g., whether a visit to a medical institution was made, whether information was shared with family) after alert issuance and using it as AI training data for future analysis. As a technical effect, the system achieves high-precision and early detection of signs of dementia by automatically analyzing vast time-series data in high-dimensional space and applying unconventional feature extraction rules (e.g., local pattern detection by CNN, long-term dependency capture by RNN), rather than relying on manual observation or interviews. This reduces the diagnostic burden on medical institutions and enables users and families to take early action proactively. Application fields include monitoring of elderly people at home, health management in care facilities, remote medical support, and corporate health management programs. Furthermore, by aggregating anonymized data from multiple users, applications for creating dementia risk maps at the regional level and public health policy are also possible.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL:https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of the above-described elements, including the collection unit, analysis unit, and alert unit, is implemented by at least one of, for example, the smart device 14 and the data processing apparatus 12. For example, the collection unit collects the user's walking patterns using a pedometer or accelerometer of the smart device 14, and collects conversation contents using speech recognition technology. In addition, the collection unit collects meal frequency using a meal recording application by a specific processing unit 290 of the data processing apparatus 12. The analysis unit analyzes the data collected by, for example, the specific processing unit 290 of the data processing apparatus 12 and detects signs of dementia using AI. The alert unit issues an alert using, for example, the notification functions, email, or voice notifications of the smart device 14. The correspondence between each unit and the device or control unit is not limited to the above examples, and various modifications are possible.

### Second Embodiment

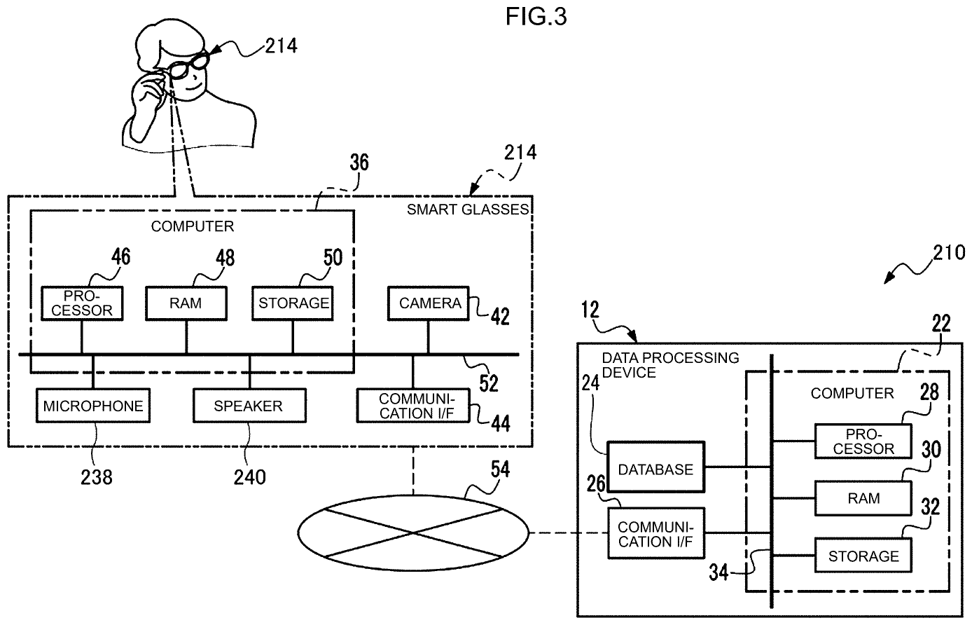

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

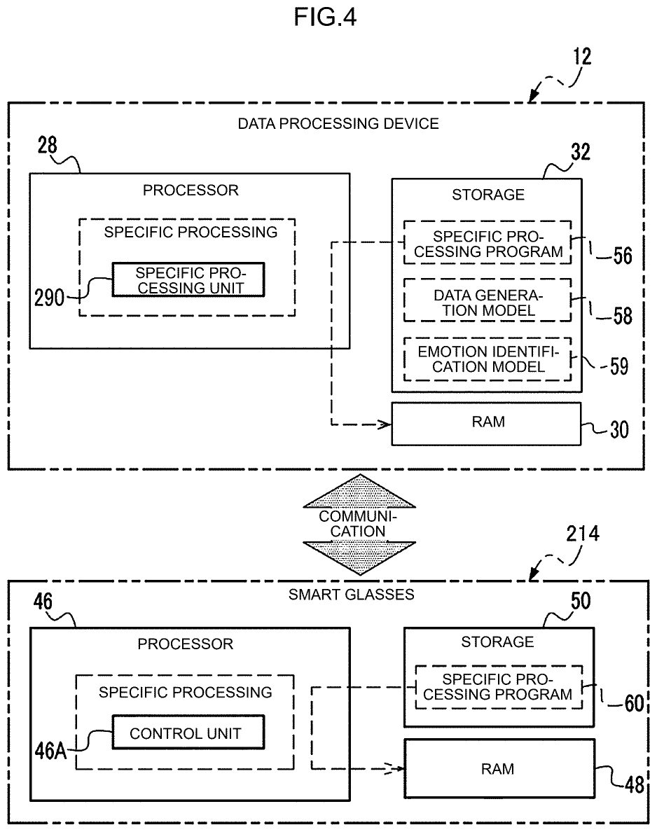

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of the above-described elements, including the collection unit, analysis unit, and alert unit, is implemented by at least one of, for example, the smart glasses 214 and the data processing apparatus 12. For example, the collection unit collects the user's walking patterns using a pedometer or accelerometer of the smart glasses 214, and collects conversation contents using speech recognition technology. In addition, the collection unit collects meal frequency using a meal recording application by a specific processing unit 290 of the data processing apparatus 12. The analysis unit analyzes the data collected by, for example, the specific processing unit 290 of the data processing apparatus 12 and detects signs of dementia using AI. The alert unit issues an alert using, for example, the notification functions, email, or voice notifications of the smart glasses 214. The correspondence between each unit and the device or control unit is not limited to the above examples, and various modifications are possible.

### Third Embodiment

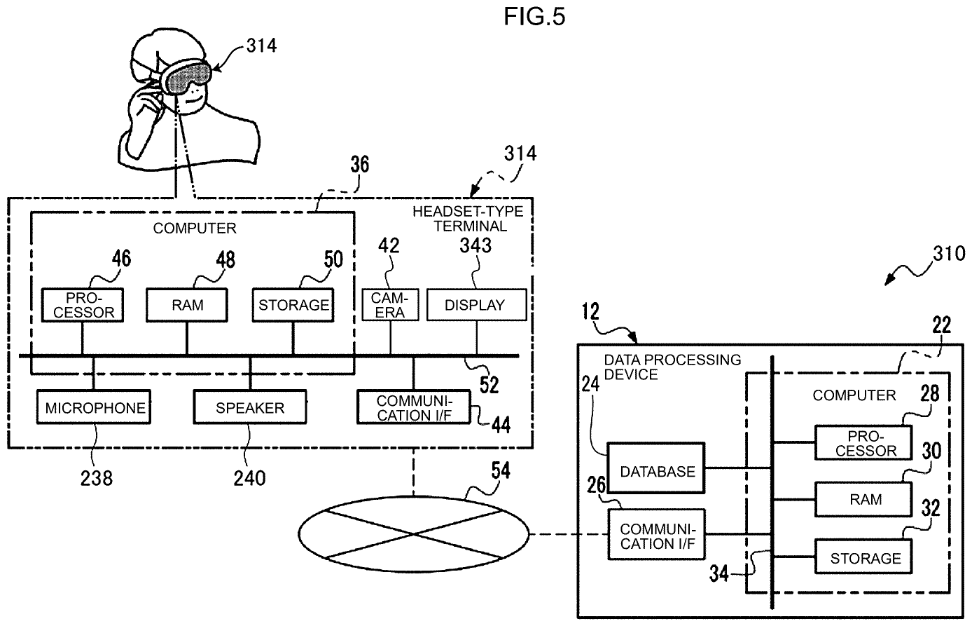

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

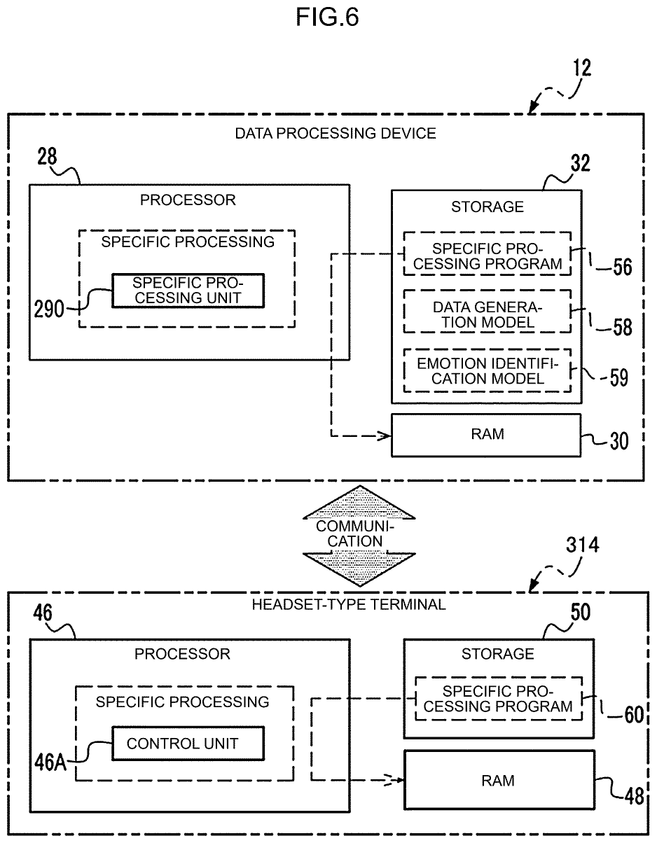

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of the above-described elements, including the collection unit, analysis unit, and alert unit, is implemented by at least one of, for example, the headset-type terminal 314 and the data processing apparatus 12. For example, the collection unit collects the user's walking patterns using a pedometer or accelerometer of the headset-type terminal 314, and collects conversation contents using speech recognition technology. In addition, the collection unit collects meal frequency using a meal recording application by a specific processing unit 290 of the data processing apparatus 12. The analysis unit analyzes the data collected by, for example, the specific processing unit 290 of the data processing apparatus 12 and detects signs of dementia using AI. The alert unit issues an alert using, for example, the notification functions, email, or voice notifications of the headset-type terminal 314. The correspondence between each unit and the device or control unit is not limited to the above examples, and various modifications are possible.

### Fourth Embodiment

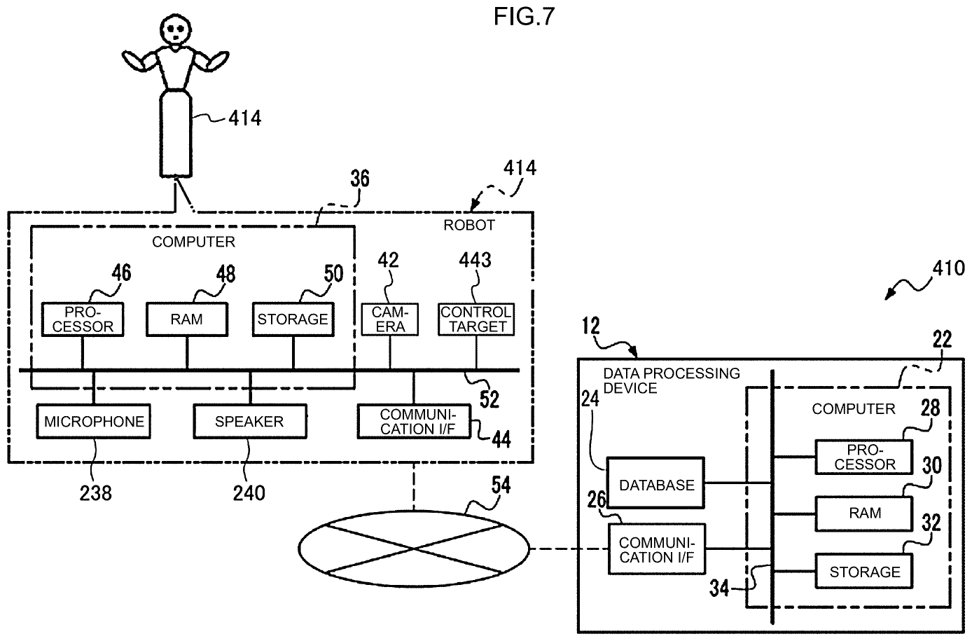

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

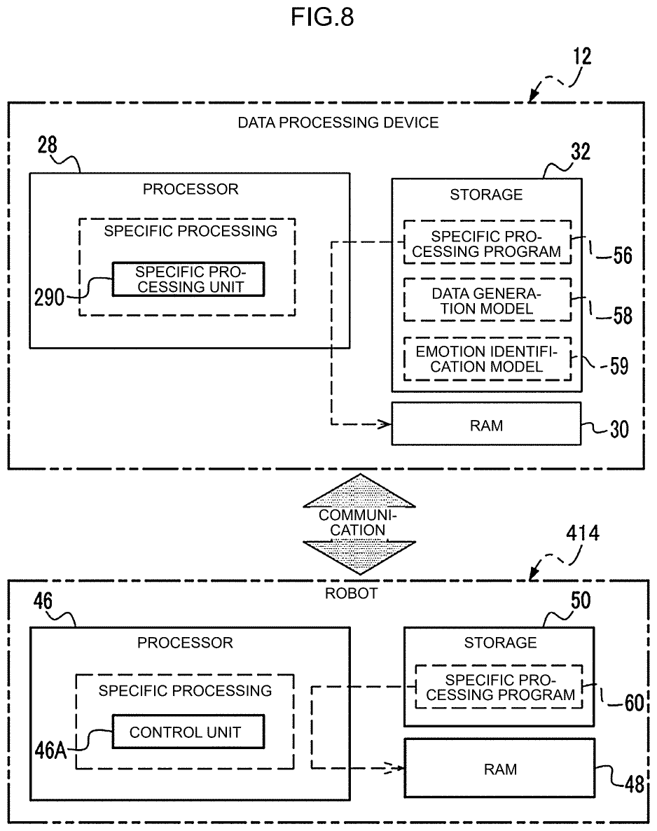

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of the above-described elements, including the collection unit, analysis unit, and alert unit, is implemented by at least one of, for example, the robot 414 and the data processing apparatus 12. For example, the collection unit collects the user's walking patterns using a pedometer or accelerometer of the robot 414, and collects conversation contents using speech recognition technology. In addition, the collection unit collects meal frequency using a meal recording application by a specific processing unit 290 of the data processing apparatus 12. The analysis unit analyzes the data collected by, for example, the specific processing unit 290 of the data processing apparatus 12 and detects signs of dementia using AI. The alert unit issues an alert using, for example, the notification functions, email, or voice notifications of the robot 414. The correspondence between each unit and the device or control unit is not limited to the above examples, and various modifications are possible.

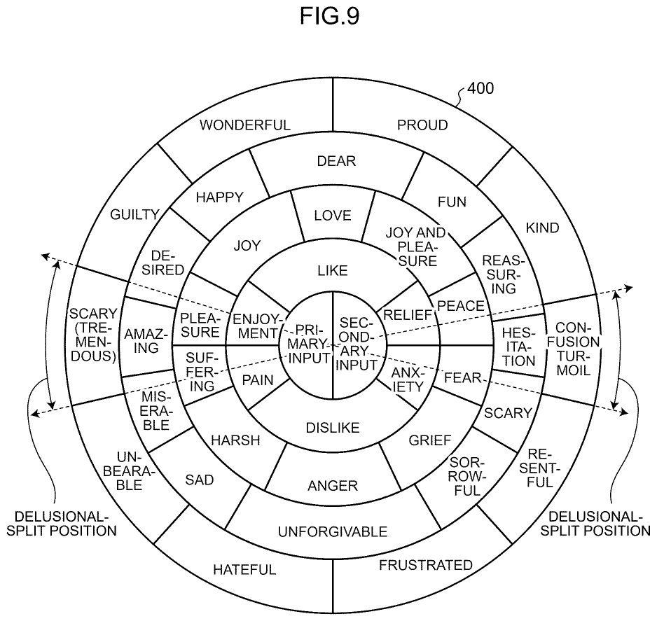

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

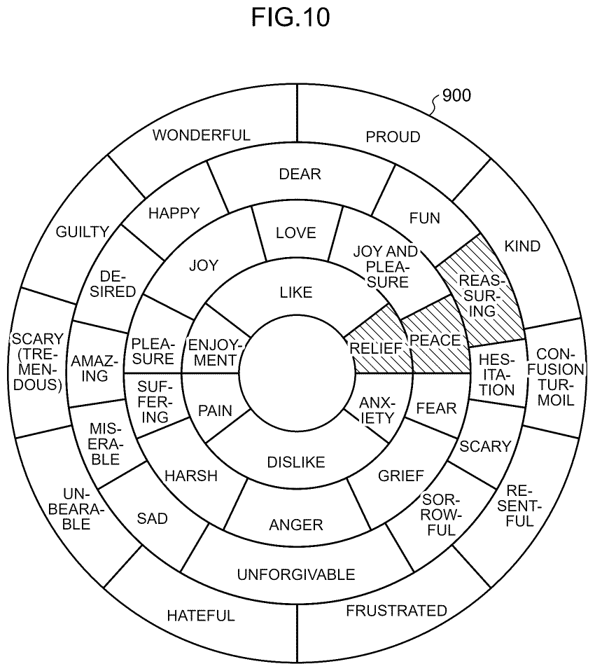

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.

(Supplementary Note 1) A system comprising: a collection unit configured to collect daily activity data of a user; an analysis unit configured to analyze the data collected by the collection unit and predict the possibility that the user corresponds to a pre-dementia state; and an alert unit configured to issue an alert based on a prediction result obtained by the analysis unit.

(Supplementary Note 2) The system according to Supplementary Note 1, wherein the collection unit is configured to collect data such as the user's walking patterns, conversation contents, and meal frequency.

(Supplementary Note 3) The system according to Supplementary Note 1, wherein the analysis unit is configured to analyze the user's behavior patterns based on the collected data and detect signs of dementia.

(Supplementary Note 4) The system according to Supplementary Note 1, wherein the alert unit is configured to issue an alert using smartphone notification functions, email, voice notifications, or the like.

(Supplementary Note 5) The system according to Supplementary Note 1, wherein the collection unit anonymizes the collected data and does not provide it to third parties.

(Supplementary Note 6) The system according to Supplementary Note 1, wherein the collection unit is configured to estimate the user's emotions and adjust the timing of collecting behavior data based on the estimated emotions.

(Supplementary Note 7) The system according to Supplementary Note 1, wherein the collection unit is configured to analyze the user's past behavior data and select an optimal collection method.

(Supplementary Note 8) The system according to Supplementary Note 1, wherein the collection unit is configured to perform filtering based on the user's current health condition and living environment when collecting behavior data.

(Supplementary Note 9) The system according to Supplementary Note 1, wherein the collection unit is configured to estimate the user's emotions and determine the priority of behavior data to be collected based on the estimated emotions.

(Supplementary Note 10) The system according to Supplementary Note 1, wherein the collection unit is configured to consider the user's geographic location information when collecting behavior data and preferentially collect highly relevant data.

(Supplementary Note 11) The system according to Supplementary Note 1, wherein the collection unit is configured to analyze the user's social media activities when collecting behavior data and collect relevant data.

(Supplementary Note 12) The system according to Supplementary Note 1, wherein the analysis unit is configured to estimate the user's emotions and adjust the expression method of analysis based on the estimated emotions.

(Supplementary Note 13) The system according to Supplementary Note 1, wherein the analysis unit is configured to adjust the level of detail of analysis based on the importance of behavior data during analysis.

(Supplementary Note 14) The system according to Supplementary Note 1, wherein the analysis unit is configured to apply different analysis algorithms according to the category of behavior data during analysis.

(Supplementary Note 15) The system according to Supplementary Note 1, wherein the analysis unit is configured to estimate the user's emotions and adjust the length of analysis based on the estimated emotions.

(Supplementary Note 16) The system according to Supplementary Note 1, wherein the analysis unit is configured to determine the priority of analysis based on the timing of collecting behavior data during analysis.

(Supplementary Note 17) The system according to Supplementary Note 1, wherein the analysis unit is configured to adjust the order of analysis based on the relevance of behavior data during analysis.

(Supplementary Note 18) The system according to Supplementary Note 1, wherein the alert unit is configured to estimate the user's emotions and adjust the alert issuing method based on the estimated emotions.

(Supplementary Note 19) The system according to Supplementary Note 1, wherein the alert unit is configured to adjust the level of detail of alerts based on the importance of the prediction result when issuing an alert.

(Supplementary Note 20) The system according to Supplementary Note 1, wherein the alert unit is configured to apply different alert issuing means according to the user's attribute information when issuing an alert.

(Supplementary Note 21) The system according to Supplementary Note 1, wherein the alert unit is configured to estimate the user's emotions and determine the priority of alerts based on the estimated emotions.

(Supplementary Note 22) The system according to Supplementary Note 1, wherein the alert unit is configured to consider the user's geographic location information and select an optimal alert issuing means when issuing an alert.

(Supplementary Note 23) The system according to Supplementary Note 1, wherein the alert unit is configured to analyze the user's social media activities and adjust the content of alerts when issuing an alert.

What is claimed is:

1. A system comprising:
a communication interface configured to communicate with a client terminal via a packet-switched network;
a memory storing a data generation model obtained by deep learning on a neural network, and an emotion identification model;
a database; and
circuitry configured to:
receive, from the client terminal via the communication interface, activity data comprising at least one of time-series motion data, text data, or event data, and store the activity data in the database;
analyze the activity data stored in the database using a time-series prediction model comprising at least one of a recurrent neural network or a long short-term memory network to extract a pattern feature vector;
compute, using the data generation model, a risk score based on the pattern feature vector and reference data stored in the database;
estimate an emotion of a user by applying the emotion identification model to sensor data received from the client terminal via the communication interface;
generate, using the data generation model, inference data comprising at least one of a notification text or a recommended action, based on the risk score and the estimated emotion; and
transmit the inference data to the client terminal via the communication interface and the packet-switched network, the inference data causing the client terminal to present the inference data to the user.

2. The system according to claim 1, wherein the activity data comprises at least one of walking pattern data acquired from an accelerometer, conversation data converted from speech by a speech recognition engine, or meal event data from a meal recording application.

3. The system according to claim 1, wherein the time-series prediction model further comprises a Transformer-based model with a self-attention mechanism, and wherein the circuitry is configured to extract the pattern feature vector by processing the activity data as a multidimensional time-series tensor.

4. The system according to claim 1, wherein the risk score indicates a probability of a pre-dementia state, and wherein the circuitry is further configured to generate the inference data comprising a recommendation to visit a medical institution when the risk score exceeds a predetermined threshold.

5. The system according to claim 1, wherein the circuitry is further configured to adjust a timing of receiving the activity data from the client terminal based on the estimated emotion, such that when the estimated emotion indicates stress, the circuitry reduces a frequency of receiving the activity data, and when the estimated emotion indicates relaxation, the circuitry increases the frequency.

6. The system according to claim 1, wherein the circuitry is further configured to analyze a collection history stored in the database associated with the user to select an optimal data collection method using at least one of a decision tree, a random forest, or a reinforcement learning model.

7. The system according to claim 1, wherein the circuitry is further configured to receive, from the client terminal via the communication interface, attribute data of the user comprising at least one of a health condition or a living environment, and to filter the activity data based on the attribute data.

8. The system according to claim 1, wherein the circuitry is further configured to determine a priority of the activity data to be received based on the estimated emotion, such that when the estimated emotion indicates stress, the circuitry prioritizes activity data having a high importance attribute.

9. The system according to claim 1, wherein the circuitry is further configured to receive geographic location information from the client terminal via the communication interface, and to preferentially receive activity data associated with a geographic region corresponding to the geographic location information.

10. The system according to claim 1, wherein the circuitry is further configured to receive social media activity data from the client terminal via the communication interface, analyze the social media activity data using a natural language processing model to extract relevance scores, and adjust a priority of the activity data to be received based on the relevance scores.

11. The system according to claim 1, wherein the circuitry is further configured to adjust an expression method of the inference data based on the estimated emotion, such that when the estimated emotion indicates stress, the circuitry generates the inference data in a concise format, and when the estimated emotion indicates relaxation, the circuitry generates the inference data in a detailed format.

12. The system according to claim 1, wherein the circuitry is further configured to calculate an importance score for each item of the activity data and to adjust a level of detail of analysis based on the importance score, such that detailed analysis is performed for activity data having a high importance score and simplified analysis is performed for activity data having a low importance score.

13. The system according to claim 1, wherein the circuitry is further configured to apply different analysis algorithms according to a category of the activity data, such that for the time-series motion data, the circuitry applies an anomaly detection algorithm, and for the text data, the circuitry applies a natural language processing algorithm.

14. The system according to claim 1, wherein the circuitry is further configured to determine a priority of analysis based on a timestamp associated with the activity data, such that activity data having a more recent timestamp is analyzed with a higher priority.

15. The system according to claim 1, wherein the circuitry is further configured to adjust a method of transmitting the inference data to the client terminal based on the estimated emotion, the method comprising at least one of a push notification, an email notification, or a voice notification.

16. The system according to claim 1, wherein the circuitry is further configured to receive attribute information of the user comprising at least one of age, occupation, or device usage status, and to select an optimal notification means for transmitting the inference data based on the attribute information.

17. The system according to claim 1, wherein the circuitry is further configured to determine a priority of the inference data based on the estimated emotion, such that when the estimated emotion indicates stress, the circuitry prioritizes inference data having a high importance attribute.

18. A system comprising:
a communication interface configured to communicate, via a packet-switched network conforming to at least one of a 5G, Wi-Fi, or Bluetooth communication standard, with a client terminal comprising a touch panel, a microphone, a speaker, a camera having a CMOS image sensor, and a display;
a processor;
a random-access memory;
a memory storing a data generation model obtained by deep learning on a neural network, and an emotion identification model;
a database; and
circuitry configured to:
receive, from the client terminal via the communication interface, activity data comprising at least one of time-series motion data from an accelerometer, text data converted from speech captured by the microphone, or event data, and store the activity data in the database;
analyze the activity data stored in the database using a time-series prediction model comprising at least one of a recurrent neural network, a long short-term memory network, or a Transformer-based model to extract a pattern feature vector;
compute, using the data generation model, a risk score based on the pattern feature vector and reference data stored in the database;
estimate an emotion of a user by applying the emotion identification model to at least one of voice data captured by the microphone or image data captured by the camera;
generate, using the data generation model, inference data comprising at least one of a notification text or a recommended action, based on the risk score and the estimated emotion;
adjust at least one of a format, a level of detail, or a notification method of the inference data based on the estimated emotion; and
transmit the inference data to the client terminal via the communication interface, the inference data causing the client terminal to present the inference data to the user via at least one of the display or the speaker.

19. The system according to claim 18, wherein the data generation model comprises at least one of a text generation AI, an image generation AI, or a multimodal generation AI, and wherein the data generation model is a fine-tuned model configured to output inference results from prompts without instructions.

20. A method performed by circuitry of a data processing system comprising a communication interface, a memory storing a data generation model obtained by deep learning on a neural network and an emotion identification model, and a database, the method comprising:
receiving, from a client terminal via the communication interface and a packet-switched network, activity data comprising at least one of time-series motion data, text data, or event data, and storing the activity data in the database;
analyzing the activity data stored in the database using a time-series prediction model comprising at least one of a recurrent neural network or a long short-term memory network to extract a pattern feature vector;
computing, using the data generation model, a risk score based on the pattern feature vector and reference data stored in the database;
estimating an emotion of a user by applying the emotion identification model to sensor data received from the client terminal via the communication interface;
generating, using the data generation model, inference data comprising at least one of a notification text or a recommended action, based on the risk score and the estimated emotion; and
transmitting the inference data to the client terminal via the communication interface and the packet-switched network, the inference data causing the client terminal to present the inference data to the user.
